(19) United States
(12) Patent Application Publication
(10) Pub. No.: US 2009/0275960 A1
(43) Pub. Date: Nov. 5, 2009

(54) APPARATUS AND METHOD FOR GASTRIC REDUCTION

(76) Inventors: John Mark Provenza, Shreveport, LA (US); Barrett Hutto, Santa Cruz, CA (US); Philip Trenholme, Santa Cruz, CA (US)

Correspondence Address: CARR LLP, 670 FOUNDERS SQUARE, 900 JACKSON STREET, DALLAS, TX, 75202, US

(21) Appl. No.: 12/420,759

(22) Filed: Apr. 8, 2009

Related U.S. Application Data
(60) Provisional application No. 61/043,178, filed on Apr. 8, 2008.

(51) Int. Cl. A61B 17/12; A61B 17/08; A61B 17/04

(52) U.S. Cl. 606/144; 606/153; 606/228

(57) ABSTRACT

An apparatus and method for gastric reduction is provided comprising a tissue gathering assembly for applying a force to inner portions of a stomach to collapse the inner portions toward the tissue gathering assembly, and a suture assembly moveably mounted to the tissue gathering assembly for advancing a suture into the inner portions to form a gastric sleeve. The tissue gathering assembly may further comprise a vacuum shaft having sections of apertures, wherein each section transmits a vacuum force through the apertures to gather a part of the inner portions of the stomach to form a channel that connects an upper pouch to a lower pouch of the stomach. The suture assembly may further comprise a rotatable helical suture for fastening the gathered inner portions to secure the gastric sleeve.

### CROSS-REFERENCE TO RELATED APPLICATIONS

This application relates to, and claims the benefit of the filing date of, co-pending U.S. provisional patent application Ser. No. 61/043,178 entitled GASTRIC REDUCTION DEVICE AND METHOD, filed Apr. 8, 2008, the entire contents of which are incorporated herein by reference for all purposes.

### FIELD OF THE INVENTION

This invention relates to the reduction of gastric volume in order to treat obesity and, in particular, to non-surgical devices and methods for reduction in the size of the stomach by forming a gastric sleeve within the stomach that regulates the passage of food material.

### BACKGROUND TO THE INVENTION

Bariatric surgical procedures have become a common surgical solution when other treatment approaches in the treatment of obesity and obesity-related comorbidities have failed. Often the patient has already tried to control or reduce weight by non-surgical approaches such as changes in diet, pharmaceuticals, psychological treatment, and exercise. For a patient, surgical techniques are a last resort. Bariatric surgery can be divided into one category that primarily causes malabsorption and another that primarily restricts gastric volume.

In the first category, open gastric bypass procedures cause malabsorption by creating a small gastric pouch that is formed by stapling or transecting the stomach. The pouch is connected to a section of the jejunum that is pulled up to the pouch as a Roux-en-Y limb. Digestive enzymes do not reach a portion of ingested foodstuffs which have been diverted through the Roux-en-Y limb. Thus, the degree of malabsorption depends on the length of the limb and its capacity to delay the digestion process.

As a surgical procedure, a gastric bypass exposes the patient to many specialized risks. These include pulmonary embolism, hemorrhaging, infection, splenic injury, ulcers, hernias, anastomotic leaks, and nutrient deficiencies. The same procedure can be accomplished by laparoscopic gastric bypass surgery which though not reducing some of the major complications or costs does reduce the risk of infections, hernias, longer hospital stays, recovery time, and overall low quality of life.

In the second category, gastric banding surgery reduces gastric size generally by wrapping a band around the upper stomach. In one type of banding surgery, laparoscopic adjustable gastric banding (LAGB), the band is inserted through small incisions in the patient's torso. The size of the band can be adjusted through a subcutaneously implanted port where saline can be injected or withdrawn to change the size of the band. By increasing or decreasing band circumference, the rate of weight loss can be varied.

In another type of banding surgery, vertical banding gastroplasty (VBG) a combination of staples and a band are used to create a pouch in the stomach. Like LAGB, the stomach is accessed by the operator laparoscopically through several small incisions in combination with laparoscopes.

Banding surgeries reduce the risk of major complications when compared to the gastric bypass procedure. However, they still require a hospital stay, though reduced, and as an invasive procedure still introduce some of the complications associated with open surgery. Some unique complications such as band slippage, silicon leakage, band erosion, port displacement, port disconnection and infections can occur.

What is needed is a non-invasive, outpatient alternative procedure which reduces trauma to the patient, minimizes the risks of complications, and provides for faster recovery time while at the same time giving the patient the same potential for weight loss that other procedures have.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 illustrates a first gastric reduction device;

FIG. 2 is a cross-sectional view of a portion of the first gastric reduction device 100;

FIG. 3 is a partial section view of a needle housing;

FIG. 4A is a perspective view of an outer rotational shaft coupled to a needle suture;

FIG. 4B is a perspective view of an outer rotational shaft coupled to a needle suture in position to engage a needle housing;

FIGS. 5A and 5B are a perspective views of a helical needle and a suture;

FIGS. 5C and 5D are close-up views of a tip of a helical needle;

FIG. 6A illustrates a second gastric reduction device;

FIG. 6B is a cross-section taken along the lines 6B-6B in FIG. 6A;

FIGS. 7 and 8 are exploded views of components of a second gastric reduction device;

FIG. 9 is a cross-sectional view of a second gastric reduction device body taken along the lines 9-9 in FIG. 6A;

FIG. 10 is a cross-sectional view of a handle assembly taken along the lines 6B-6B in FIG. 6A, and the detail 10 in FIG. 6B;

FIG. 11 is a perspective view of a handle member and a first insert;

FIGS. 12A and 12B are rear and side views, respectively, of a handle member;

FIGS. 13A and 13B are side and front views, respectively, of a first insert;

FIG. 14 is a perspective view of an actuator and a second insert;

FIGS. 15A and 15B are rear and side views, respectively, of an actuator;

FIGS. 16A and 16B are rear and side views of a second insert;

FIG. 17 is a perspective view of a suture assembly;

FIG. 18 is a cross-sectional view of a suture assembly taken along the lines 6B-6B in FIG. 6A, and the detail 18 in FIG. 6B;

FIGS. 19A and 19B are side and end views, respectively, of a suture connector;

FIGS. 20A and 20B are side and end views, respectively, of a helical suture;

FIG. 21 is a perspective view of a vacuum shaft;

FIGS. 22A and 22B are a side view and a bottom view, respectively of a vacuum shaft;

FIGS. 23A-D illustrates a first, a second, a third, and fourth operational position, respectively, for a second gastric reduction device;

FIGS. 24A and 24B are perspective views of other implements used in conjunction with a gastric reduction device; and

FIGS. 25-34 illustrates operations in a method for gastric reduction.

### SUMMARY OF THE INVENTION

These and other objects and advantages are achieved in accordance with an embodiment of the present invention, wherein at least an apparatus and a method for reducing gastric volume is provided comprising a tissue gathering assembly for applying a force to inner portions of a stomach to collapse the inner portions toward the tissue gathering assembly, and a suture assembly moveably mounted to the tissue gathering assembly for advancing the suture assembly into the inner portions to form a gastric sleeve.

### DETAILED DESCRIPTION

In the following discussion, numerous specific details are set forth to provide a thorough understanding of the present invention. However, those skilled in the art will appreciate that the present invention may be practiced without being limited to such specific details.

### First Gastric Reduction Device 100

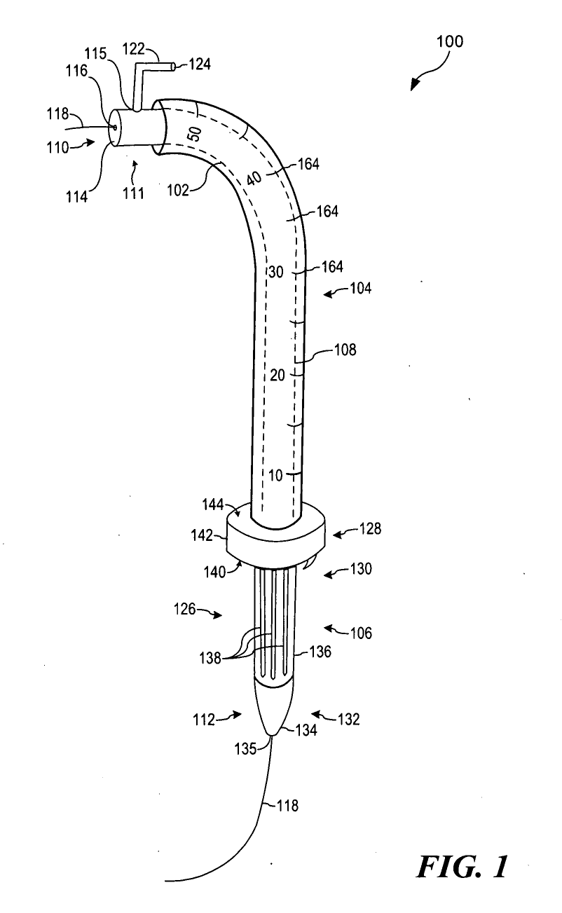

Referring to FIG. 1, the embodiment shown refers to a first gastric reduction device 100 which may be delivered transesophageally over a guide wire 118 and with the assistance of an endoscope or other method of indirect observation of internal organs. In the embodiment shown, the first gastric reduction device 100 may be comprised of an inner vacuum shaft 102 and an outer rotational shaft 104, whereby the outer rotational shaft 104 may be generally a cylindrical shape and at least partially fit over the inner vacuum shaft 102 which also may have a generally cylindrical shape. The inner vacuum shaft 102 may also couple to the outer rotational shaft 104. When coupled to the inner vacuum shaft 102, the outer rotational shaft 104 may be configured to rotate relative to the inner vacuum shaft 102. The embodiments referred to herein will be referred to as “proximal” to the operator and the mouth portion of the patient. The term “distal” will refer to a position extending away from the operator or mouth portion of the patient.

The inner vacuum shaft 102 may fit at least partially within the outer rotational shaft 104 and may have an exposed distal portion 106 and a proximal end 110 that comprises an exposed portion 111. At the exposed distal portion 106 and the exposed portion 111 of the proximal end 110 the inner vacuum shaft may extend out and remain uncovered by the outer rotational shaft 104.

### Inner Vacuum Shaft 102

In the embodiment shown in FIG. 1, the inner vacuum shaft 102 may at least partially comprise an elongated tube portion 108 having a generally cylindrically shape with enough longitudinal length to pass through the mouth of the patient and reach the stomach of the patient. The inner vacuum shaft 102 may further comprise a proximal end 110 and a distal end 112 and be made from a moldable and flexible material, such as plastic. It should be apparent that the inner vacuum shaft 102 may be made from other alternative materials readily known by persons of ordinary skill in the art and that these materials may be interchangeable.

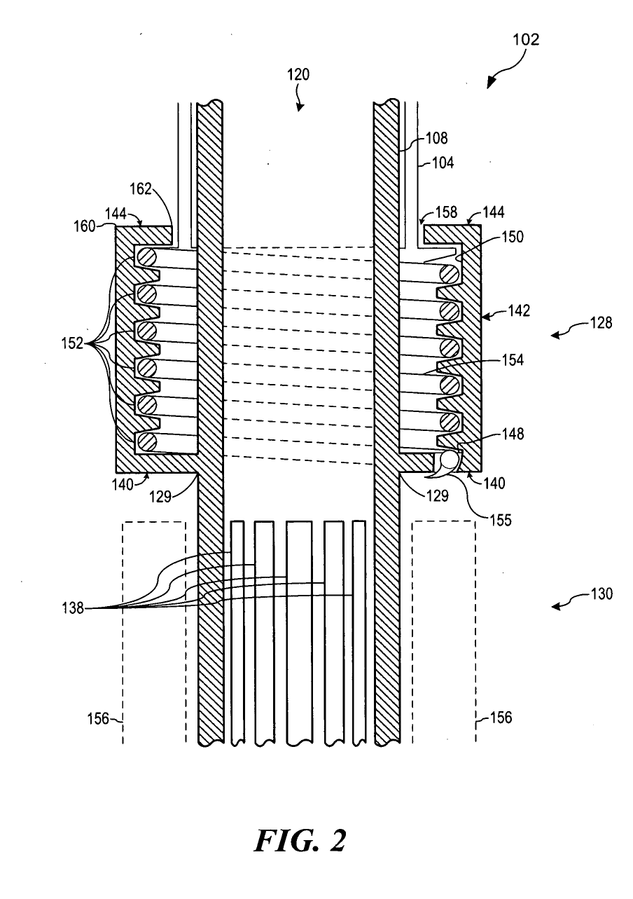

Being hollow, the inner vacuum shaft 102 may form a cavity 120 (FIG. 2) comprising a channel that runs longitudinally from the proximal end 110 of the inner vacuum shaft 102 to the distal end 112 of the inner vacuum shaft 102.

The exposed portion 111 of the proximal end 110 of the inner vacuum shaft 102 may comprise a capped portion 114 where the channel comprising cavity 120 may be at least partially capped. The capped portion 114 may comprise a guide wire opening 116 where the guide wire 118 may pass into the cavity 120 (not shown in FIG. 1) of the inner vacuum shaft 102. The guide wire opening 116 may be shaped to allow the guide wire 118 to fit snugly and provide a seal which prevents air leakage and degradation in vacuum force.

The guide wire 118 may comprise a wire designed to pass through a guide wire tube 119 (not shown) in the gastric reduction device from the proximal end 110 at the guide wire opening 116 of the device 100 through the distal end 112. Such guide wire and tube configurations are readily known in the art and it would be apparent that persons of ordinary skill would recognize that that such means may be used to configure the first gastric reduction device 100 to allow passage by means of a guide wire. In addition, the guide wire 118 may be utilized to pass an endoscopic device to allow direct observation of the internal organs of the patient.

The guide wire tube 119 should be sealed at the guide wire opening 116 and the guide wire exit 135. The technique of passing surgical instrument over a guide wire is well-known in the art. The guide wire may assist the operator in the passage and placement of the first gastric reduction device 100.

Also, a suction port opening 115 may be located on the exposed portion 111 of the proximal end 110 of the inner vacuum shaft 102. The suction port opening 115 may be an opening in the exposed portion 111 of the proximal end 110 configured to fit a suction port 122. The suction port 122 may be a small tube. The suction port 122 may be detachable to allow the fitting of the outer rotational shaft 104 over the inner vacuum shaft 102. The suction port 122 may then couple to the exposed portion 111 of the proximal end 110 at the suction port opening 115.

Also, the proximal end 110 of the inner vacuum shaft 102 may further comprise the suction port 122. The suction port 122 may be comprised of a port opening 124. The suction port 122 at the port opening 124 may be configured to couple to a vacuum source (not shown).

The suction port 122 and the cavity 120 of the inner vacuum shaft 102 may allow the flow of air to pass from the proximal end of the inner vacuum shaft 102 to the vacuum source. Thus, from openings in the distal end 112 of the inner vacuum shaft 102 suction force can be applied to the inner walls of the stomach.

### Suction Tube 126

The exposed distal end 112 of the inner vacuum shaft 102 may comprise a suction tube 126 and a needle housing 128. The suction tube 126 comprises an exposed portion of the inner vacuum shaft 102 which extends away from the proximal end 110 of the inner vacuum shaft 102. The suction tube 126 further comprises a proximal end 130 and a distal end 132.

The suction tube 126 is further comprised of a tube tip 134 and a tube body 136. Generally, the suction tube body 136 may be substantially hollow and cylindrical in shape. The substantial cylindrical shape may allow for tissue to be gathered by suction around the outside of the tube and may allow the formation of a gastric sleeve of uniform diameter. The diameter of the tube body 136 may be predetermined and preconfigured as a factor in the reduction of gastric volume.

The tube tip 134 may be a hollow member tapered in a generally conical shape pointing in the direction of the distal end 132 of the suction tube 132. The tube tip 134 may also comprise a guide wire exit 135 where the guide wire tube 119 (not shown) which holds the guide wire 118 runs up to the guide wire opening 116. The guide wire exit 135 may be sealed from the interior of the tube tip 134 to prevent vacuum air from leaking. The tube body 136 extends from the proximal end 130 of the suction tube 126 to the tube tip 134. The substantially conical shape of the tube tip 134 provides for ease of insertion of the apparatus into the esophagus and into the stomach of the patient.

The tube body 136 may further comprise a plurality of slots 138. Each slot of the plurality of slots comprises an opening or aperture in the tube body 136 which may allow suction force to draw air, fluids and tissue towards the first gastric reduction device 100 and particularly toward the tube body 136.

Also, each slot of the plurality of slots 138 may be substantially evenly spaced from other slots around the tube body. Each slot of the plurality of slots 138 may have a substantially uniform width. The substantially even spacing and substantially uniform width of the plurality of slot may allow for the even suction of tissue along the tube body long axis and around the outside of the suction tube body 136. Such even suction may allow for substantially uniform distribution and binding of tissue. This embodiment then may allow for the creation of a predetermined gastric sleeve of substantially uniform diameter and also prevent the tissue that forms the gastric sleeve from tearing or slipping from its binding.

### Needle Housing 128

Referring now to FIG. 2, there is shown a cross-sectional view of a portion of the first gastric reduction device 100. The needle housing 128 may be located adjacent to the proximal end 130 of the suction tube 126 and may be meet the proximal end 130 of the suction tube 126 at edge 129. The needle housing 128 may house a binding element or a suture assembly, such as a suture needle, and may transport the binding element with the suction tube 126 as it is passed into the stomach. In some embodiments, the suture needle then may comprise a helical needle 154 further comprising multiple turns 157. Thus, the needle housing 128 may be positioned so that the suture needle may be released towards the exposed distal end 112 of the inner vacuum tube 102.

The needle housing 128 may be comprised of an insertion surface 140, a side surface 142, a withdrawal surface 144, a suture needle entrance 146, a suture needle release opening 148, an inner surface 150, and a plurality of needle grooves 152. The needle housing 128 may be generally shaped as a substantially closed cylinder sized to be wider than the diameter of the inner vacuum shaft 102 or the outer rotational shaft 104. Also, the needle housing 128 may be substantially hollow so that a needle with a helical shape fits within the needle housing 128 and can be released without interference from other structure.

The elongated tube portion 108 passes through the hollow portion of the needle housing 128 so that suctioned air may flow through cavity 120 from the plurality of slots 138 in the tube body 136 up to the suction port 122. It is to be understood that the elongated tube portion 108 and the needle housing 128 may be constructed as one piece of material with each portion meeting at edge 129.

The needle housing 128 is positioned and oriented so that the helical needle 154, comprising a needle point 155, may be released from the needle housing 128 towards the distal end 132 of the suction tube 126. The needle housing 128 may be located close enough to the suction tube 126 so that when tissue is suctioned towards the suction tube 126, the helical needle 154, when released, may catch the tissue and facilitate the gastric reduction procedure, described herein. The space where the helical needle 154 may be released to engage the suctioned tissue may define a suture area 156. The insertion surface 140 comprises a suture needle release opening 148 which may be wide enough to allow the helical needle 154 to extend out of the needle housing 128.

### Withdrawal End of Needle Housing

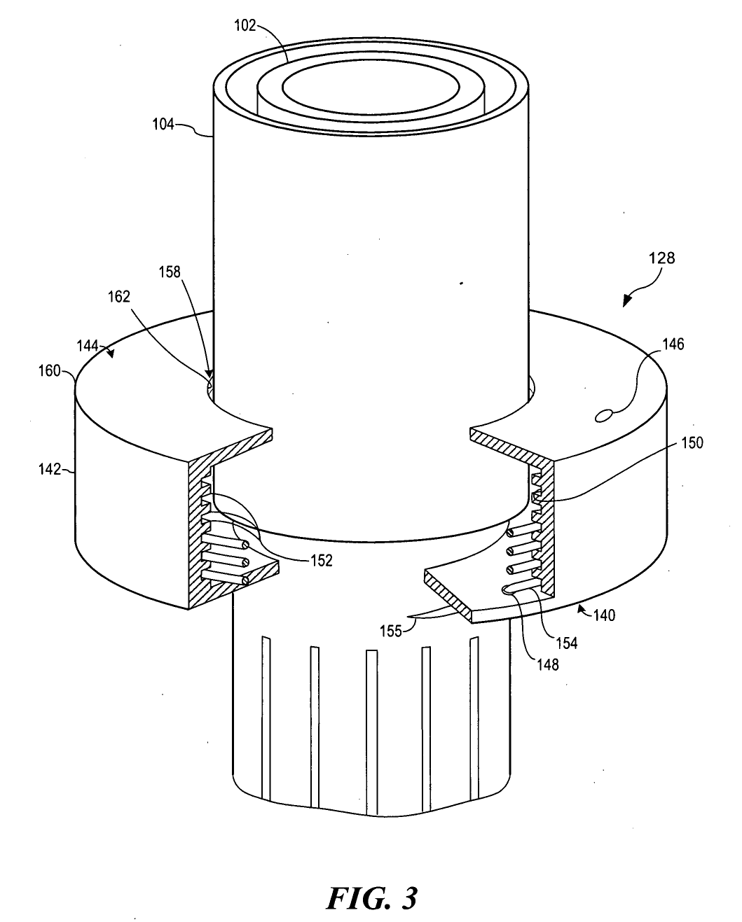

Referring to FIG. 3, there is shown a perspective view of the needle housing 128, with a portion of the housing 128 cut-away to expose portions of the helical needle 154. Located opposite the insertion surface 140 of the needle housing 128, the withdrawal surface 144 may comprise a surface which partially closes the proximal side of the needle housing and also may comprise an outer rotational shaft opening 158 and a suture needle entrance 146.

The suture needle entrance 146 may comprise an opening in the withdrawal surface 144 allowing the helical needle 154 to pass into the needle housing 128. In alternative embodiments, the suture needle may be placed into the needle housing 128 prior to placement of the withdrawal surface 146. These embodiments may eliminate the need for the suture needle entrance 146.

The outer rotational shaft opening 158 may comprise a substantially circular-shaped slot in the withdrawal surface 144 with a inner diameter that nearly matches that of the outer rotational shaft 104 and an outer diameter that may create the slot narrow enough so that the outer rotational shaft 104 fits snugly into the outer rotational shaft opening 158 but not tight enough to prevent rotation of the outer rotational shaft 104. The slot may allow at least the outer rotational shaft 104 to pass partially into the needle housing 128.

The withdrawal surface 144 may comprise a surface generally facing the proximal end 110 of the inner vacuum shaft 102. The withdrawal surface 144 may comprise a flat surface, as shown in FIG. 3, facing the direction towards the proximal end 110 of the inner vacuum shaft 102 or, in the alternative, may comprise a beveled surface (not shown). The beveled surface may comprise a sloped surface which creates a slope from an edge 160 of the side wall 142 to the edge 162 of the outer rotational shaft opening 158. The sloping of the withdrawal surface 144 may assist in the withdrawal of the device 100 from the patient by initially providing a narrower cross sectional area with less sharp edges on the side of the first gastric reduction device 100 exiting the body, thereby reducing trauma.

### Outer Rotational Shaft 104

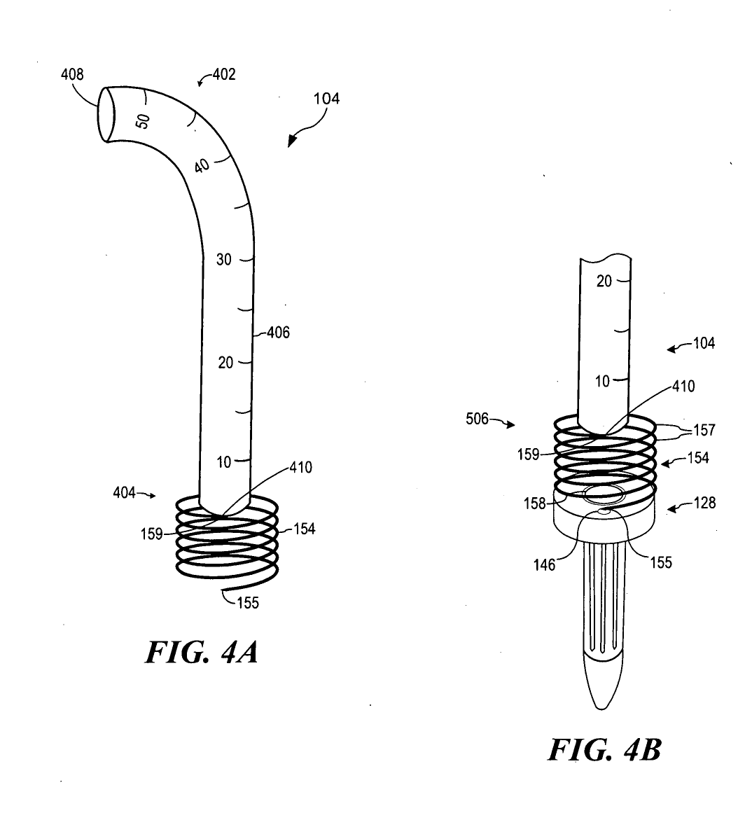

Referring now to FIG. 4A, there is shown at least one embodiment of outer rotational shaft 104 coupled to the helical needle 154. The outer rotational shaft 104 may be comprised of a proximal end 402 and a distal end 404. A tube portion 406 extends from the proximal end 402 to the distal end 404. The tube portion 406 may comprise a hollow cylindrically shaped structure. When coupled to the inner vacuum shaft 102, the tube portion 406 may surround the at least a portion of the inner vacuum shaft 102.

The proximal end 402 of the outer rotational shaft 104 comprises an opening 408 to allow the inner vacuum shaft 102 and the guide wire 118 to pass within it. In other embodiments, other instrumentation may pass though the outer rotational shaft 104 or in combination with the inner vacuum shaft 102 from the proximal end 402 of the outer rotational shaft 104 to the distal end 404.

FIG. 4B illustrates the inner vacuum shaft 102 separated from the outer rotational shaft 104. The distal end 404 of the outer rotational shaft 104 may be comprised of a connection portion 410. The outer rotational shaft 104 may slide over the inner vacuum shaft 102 covering the inner vacuum shaft 102 at least partially.

As a whole the outer rotational shaft 104 may be comprised of a uniform, flexible material such as plastic. It will be apparent to persons of ordinary skill in the art that the outer rotational shaft may be composed of a variety of materials which are suitable for medical devices.

### Installation of the Helical Needle in the Housing

Referring now to FIG. 4B, to install the helical needle 154 into the needle housing 128, the helical needle 154 may pass into the suture needle entrance 146. The helical needle 154 may comprise a needle point 155 which may pass first into the suture needle entrance.

As shown in FIG. 3, the needle housing 128 may comprise the inner surfaces 150 having needle grooves 152 which act as receptors and guides that the multiple turns 157 of the helical needle 154 may pass along. The multiple turns 157 may engage the needle grooves 152 on the inner surface 150 of the needle housing 128. The helical needle 154 may pass along each of the plurality of needle grooves 152 until the helical needle 154 is fully installed into the needle housing 128. The helical needle 154 is fully installed into the needle housing 128 when the needle point is at the suture needle release opening 148, placing the helical needle in a position for release. If coupled to the helical needle 154, the distal end 404 of the outer rotational shaft 104 may pass at least partially into the opening 158 of the withdrawal surface 144.

In some embodiments, the helical needle 154 may be precompressed prior to installation or compressed at installation to allow a helical needle with a larger length to fit within a smaller housing. The helical needle 154, if compressed, may then be released from the needle housing 128 back into its original condition.

### Helical Needle 154

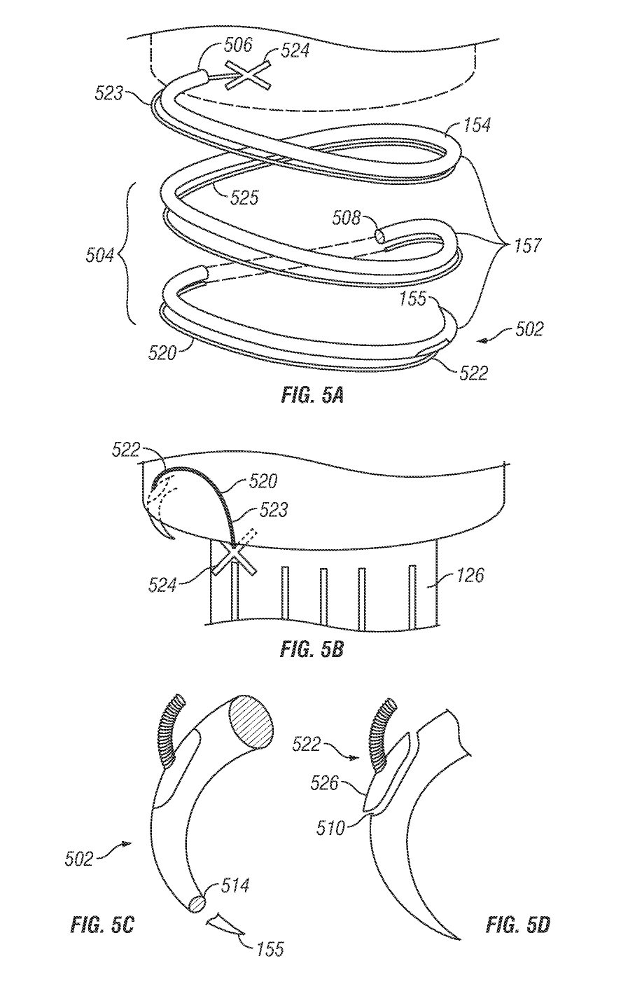

Referring now to FIGS. 5A and 5B, there are shown illustrations of the helical needle 154. The helical needle 154 may be a suture needle comprising a front portion 502, a body portion 504, and a base portion 506. The helical needle 154 may be manufactured using standard techniques already well-known in the art. The helical needle 154 may be constructed from stainless steel, though other suitable materials that are apparent to persons of ordinary skill in the art may be used.

In some embodiments, the helical needle 154 may be formed from a material, such as nitinol, which allows for spring-like compression or expansion, whereby after compression or expansion of the helical needle 154 it may return to its original condition upon the release of load. In some embodiments, this characteristic would facilitate transporting the helical needle 154 to the suture area 156 in a substantially compressed state.

The body portion 504 may be comprised of multiple turns 157 formed from the body portion 504. Each turn may comprise a coil of the helical shape comprising the helical needle. The multiple turns 157 may be spaced substantially evenly apart and sloped substantially in parallel forming a helix having a substantially constant pitch and a substantially constant radius of curvature. The multiple turns 157 may extend from the front portion 502, located at one end of the helical needle 154 to the base portion 506, located at the opposite end.

The helical shape formed by the body portion may be that of a right-handed helix or a left-handed helix. The helical needle 154 shown in FIG. 5 is generally that of a left-handed helix. Should a left-handed helical needle be required then the configuration helical needle housing 128 may be accommodated to correspond to the orientation of the multiple loops of a left handed helical needle. By example FIGS. 1, 2, 3, 4A and 4B depict right-handed helical needles.

The helical needle 154 may further comprise a cross-sectional area 508 which may be uniform across the length of the needle body 504, but may vary at the front portion 502 and the base portion 506 of the helical needle 154. The front portion 502 of the helical needle 154 may comprise a needle point 155 and a suture tie receptor 510. As shown in FIG. 5C, the needle point 155 may comprise a tapered shape wherein the cross-section 514 of the needle point 155 increases from a tip 512 to a shape to match the cross section 508 of the needle body 504. The needle point 155 may further comprise a steep point or a shallow point depending on the needs of surgery.

The length of the needle body 504 may be taken as the distance along the needle body portion from the needle point 155 to the base portion 506. The radius of curvature may be substantially uniform across the multiple loops so that the needle body portion forms a helical of substantial uniform width. The helical pitch may be defined as the spacing between the multiple loops, and the helical width may be defined as the linear distance between one outer edge of on and the helical height may be configured to fit at least partially into the needle housing 128. Further, these factors may vary according to the needs of the surgery.

Referring now to FIG. 5D, there is shown a close-up view of the tip 522 of the helical needle 154. The helical needle 154 is further comprised of a suture tie receptor 510. The suture tie receptor 510 comprises an indentation in the front portion 502 of the helical needle 154 at a location proximal to the needle point 155. The suture tie receptor 510 is configured to couple to a suture so that a suture might used to bind tissue together by the steps disclosed in this description.

Referring to FIGS. 4A and 4b, the base portion 506 of the helical needle 154 may comprise a connection portion 159. The coupling between the helical needle 154, which may be made from stainless steel, and the outer rotational shaft 104, which may be made from a moldable plastic, may be accomplished by known methods of connecting similar materials. For instance, the connection portion 159 may comprise a closed and grounded end whereby the turn of the multiple turns 157 located at the base portion 506 may has a reduced pitch and forms a plane that is generally parallel to the plane formed by the connection portion 410 of the outer rotational shaft 104. The radius of curvature of the turn of the multiple turns 157 located at the base portion 506 may also be reduced to nearly the radius of the connection portion 410 of the outer rotational shaft 104 to facilitate connection. For instance, the connection portion 159 of the helical needle 154 may sit within a curved lip of the connection portion of the outer rotational shaft 104, or the connection portion of the helical needle 154 may be imbedded into the connection portion of the outer rotational shaft 104.

### Thread Suture 520

Referring now to FIGS. 5A, 5B, 5C, and 5D, a thread suture 520 may be configured to connect to the helical needle 154 so that the thread suture 520 may bind gastric tissue gathered by the suction tube 126. Generally, the thread suture 520 may comprise, at least partially, a binding, non-absorbable material which may be strong enough to hold the gastric tissue for an indefinite time period. Such materials may include but not be limited to silk, polypropylene, polyester or nylon. The thread suture 520 may also comprise a substance which is non-toxic, hypoallergenic, coated with an anti-microbial substance, and at least semi-impermeable to fluids. Sutures made of these materials are well-known in the art and it should be apparent to persons of ordinary skill in the art that alternative embodiments fulfilling the same purposes and uses described here can be used.

In the embodiment shown in FIG. 5B, the thread suture 520 may comprise a coiled suture. Such a suture is known in the art and is comprised of a material that has been pre-selected to have a certain length, tensile strength, and elasticity. Other embodiments of prepackaged sutures may be used. It will be apparent in the art that such sutures may be interchangeable so long as the particular suture meets the needs of the procedure.

The thread suture 520 may be further comprised of a leading end 522 which may connect to the needle point 155 of the helical needle 154 and a trailing end 523 which may connect to a suture cross tie 524. Between the leading end 522 and the trailing end 523, the thread suture 520 comprises a suture body 525. The suture leading end 522 may couple to the needle point 155 by a suture tie 526, and the trailing end 523 may couple to the suction tube 126.

The leading end 522 of the thread suture 520 may comprise a suture tie 526 further comprising at least one anchor. The suture tie 526 may be made from a metallic material, such as stainless steel, in order to permanently anchor the thread suture 520 into the tissue. It will be apparent to other persons of ordinary skill in the art that the suture tie 526 may be made from other materials which will accomplish the same purpose.

The suture tie 526 is shaped to fit into the suture tie receptor 510 such that the shape is streamlined with the slope of the needle. This configuration may allow the suture tie 526 to rest in the suture tie receptor 510 as the helical needle 154 is advanced into the stomach tissue. The streamlining may prevent snagging and dragging of tissue as the helical needle 154 is advanced.

At the needle point 155, the suture tie receptor 510 may couple to the leading end 522 of the thread suture 520. A light adhesive material may be used to temporarily bind the suture tie 526 to the suture tie receptor 526. The connection may remain temporary so that when the thread suture 520 engages the gastric material, the suture tie 526 may release from the suture tie receptor 510 and the anchors may engage the gastric material, leaving the thread suture 520 in place but allowing the helical needle 154 to be retracted by the steps disclosed in this description.

The length of the thread suture 520 taken along the longitudinal axis of the suture body 525 from the leading end 522 to the trailing end 523 may be configured to be substantially the same as the length of the helical needle 154. In some embodiments the length of the thread suture 520 may be shortened to allow for suture detachment from the helical needle 124.

### Second Gastric Reduction Device 1000

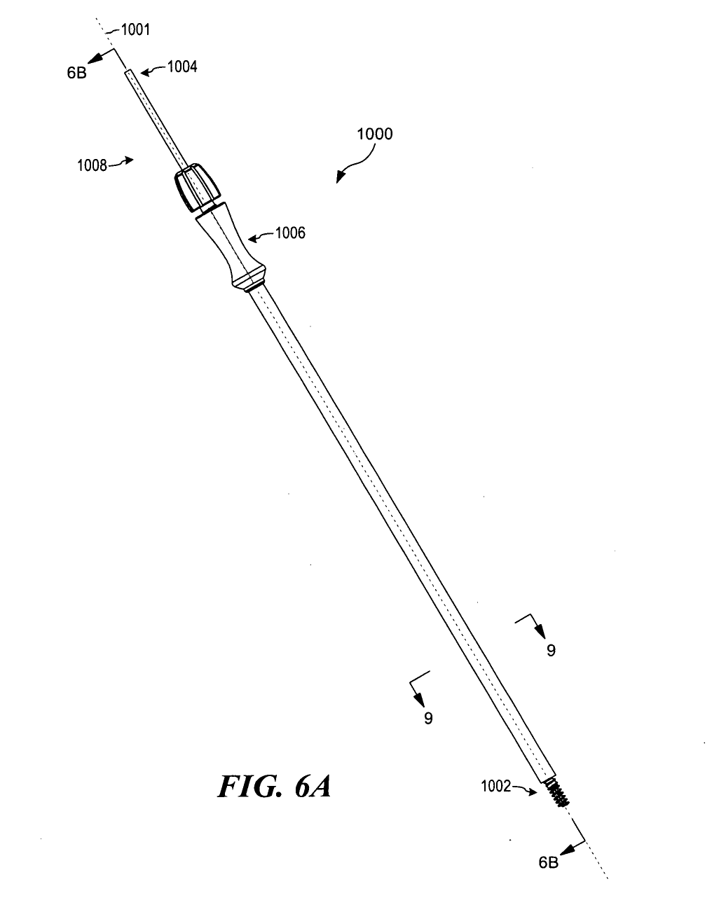

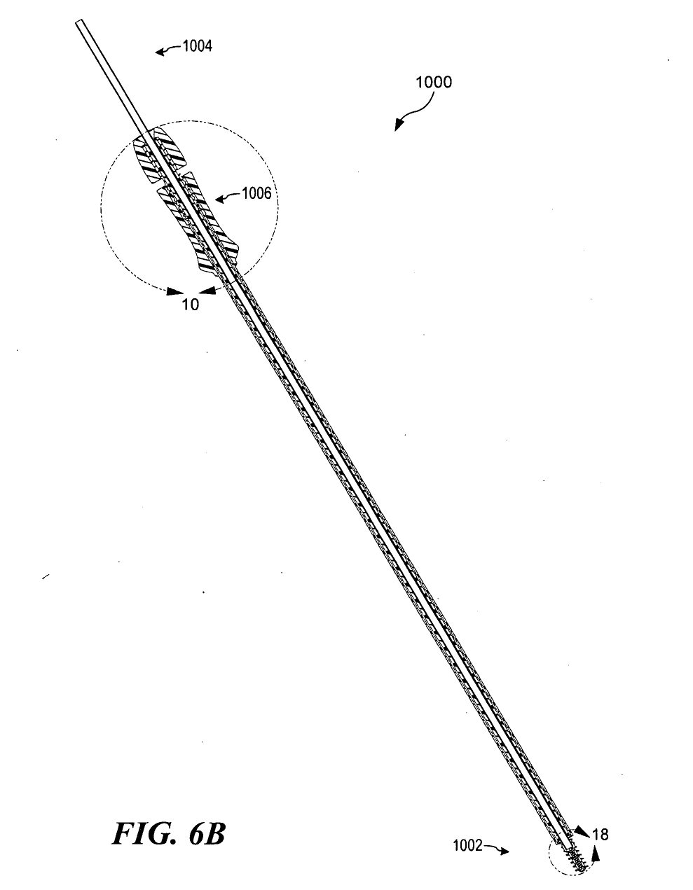

Referring now to FIG. 6A, there is shown at least one embodiment for a second gastric reduction device 1000. In FIG. 6B, there is shown a cross-section of the second gastric reduction device 1000. The second gastric reduction device 1000 may be configured to deliver a tissue binding device to the stomach for creating a sleeve that defines a channel between an upper first pouch of the stomach and a lower second pouch of the stomach. The first pouch may have generally a smaller size in relation to the second pouch so that intake of food material may be regulated for the purpose of weight loss.

The second gastric reduction device 1000 may comprise a distal end 1002 and a proximal end 1004. The distal end 1002 may be configured for insertion into an orifice or incision into a living organism, such as a human body, for accessing tissue within the body. The proximal end 1004 may be configured for operator handling and device positioning with a handle assembly 1006 and connector assembly 1008.

The second gastric reduction device 1000 may extend substantially along a device axis 1001 from the proximal end 1004 to the distal end 1002. Reference to the device axis 1001 will be made as each component of the second gastric reduction device 1000 is described.

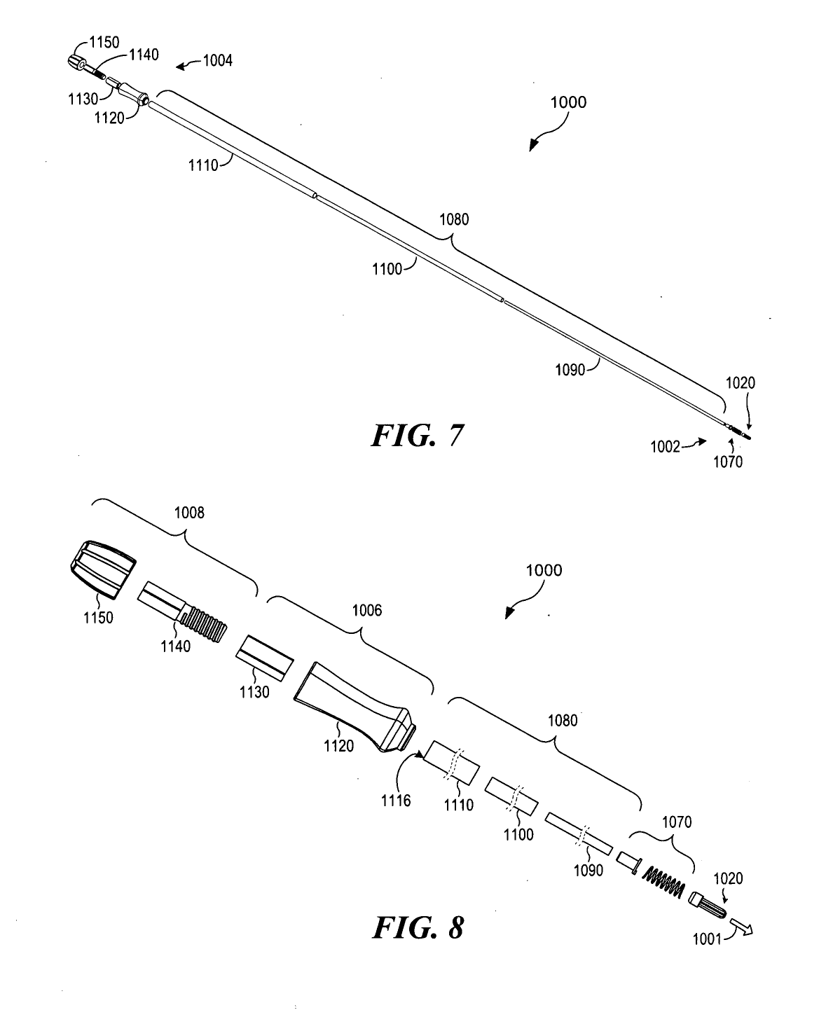

Referring now to FIGS. 7 and 8, there are shown exploded views of components of the second gastric reduction device 1000. At the distal end 1002, the second gastric reduction device 1000 may comprise a tissue gathering assembly 1020 and a suture assembly 1070. A device body 1080 of the second gastric reduction device 1000 may comprise one or more elongated tube members extending substantially along the device axis 1001, which may in some embodiments function to connect the distal end 1002 and the proximal end 1004 to position the tissue gathering assembly 1020 within the body, and to actuate the suture assembly 1070.

### Device Body 1080

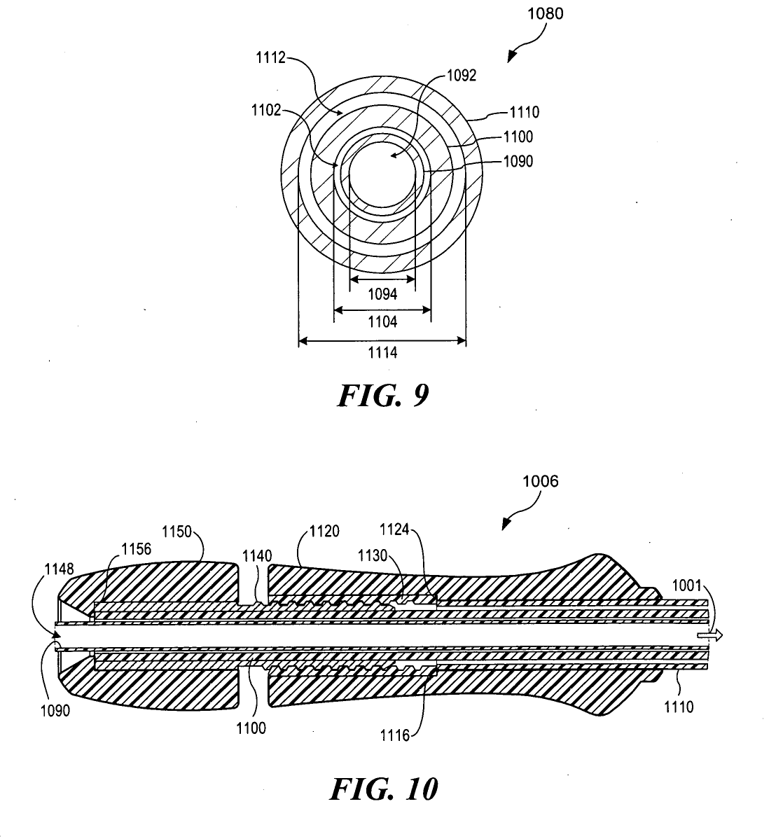

Referring now to FIG. 9, there is shown a cross-sectional view of the device body 1080 showing how each tube member fits within the other. The tube members of the body 1080 may include an inner tube 1090, a suture driver 1100, and an outer tube 1110.

The device body 1080 may be generally bendable to accommodate curvatures in an orifice or channel of the human body, such as the esophagus, the stomach, the small intestine, or the large intestine. In some embodiments, the device body 1080 may have a length configured to give manual access to an operator to reach at least a target portion of the body, such as the stomach, from an area where the operator may be free to manipulate the second gastric reduction device 1000.

The device body 1080 may comprise an inner tube 1090 for communicating a tissue gathering force, such as a suction or vacuum force. In some embodiments, the inner tube 1090 may comprise an elongated, cylindrical, and hollow tube configured to couple to the tissue gathering assembly at a proximal end and the handle assembly 1006 and connector assembly 1008 at a proximal end. The inner tube 1090 may define an inner tube channel 1092 extending from a proximal to a distal end of the inner tube 1090. A vacuum source (not shown) may be further coupled to an open end of the proximal end of the inner tube channel 1092. The inner tube 1090 may be made from a plastic, such as poly(tetrafluoroethylene) (PTFE) or other flexible chemically inert material that is commonly known by persons of ordinary skill.

The inner tube channel 1092 may comprise an inner tube channel diameter 1094, which may be configured to accommodate the amount of suction force required to gather stomach tissue to the device at the distal end of the second gastric reduction device 1000. The inner tube channel diameter 1094 may comprise the diameter of the cross-section of the inner tube channel 1092. It will be understood by persons of ordinary skill in the art that the inner tube channel diameter 1094 may be varied to vary the pressure and suction force. The inner tube channel diameter 1094 may be one factor in conjunction with other variables such as the air speed of the air passing through the channel, any attachments either at the proximal or distal end of the inner tube 1090 that may affect air flow, or any other variable commonly known by persons of ordinary skill.

The device body 1080 may further comprise a suture driver 1100 for actuating a suture 1040. In some embodiments, the suture driver 1100 may comprise an elongated corrugated tube that may receive the inner tube 1090 in a driver channel 1102 to couple to the suture 1040 for actuation at a distal end of the inner tube 1090 and to couple to the handle assembly 1006 and connector assembly 1008 (shown in FIG. 6A) at a proximal end. The driver diameter 1104 may be configured to slideably fit and accommodate the inner tube 1090 within inner walls of the driver channel 1102. There may be a clearance between the inner walls of the driver channel 1102 and outer walls of the inner tube 1090 to allow for the suture driver 1100 to rotate over the inner tube 1090.

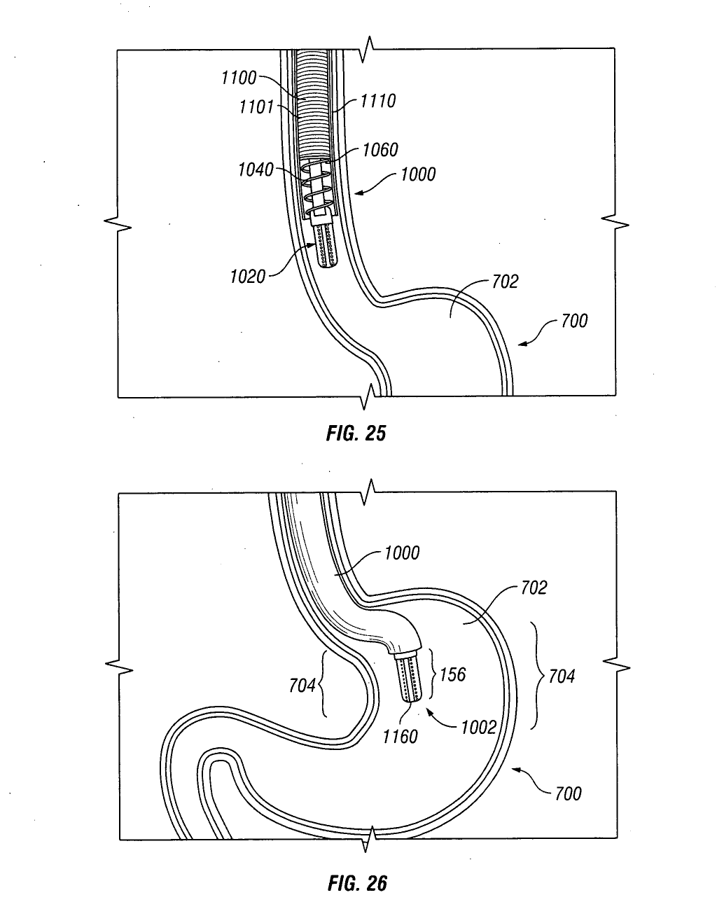

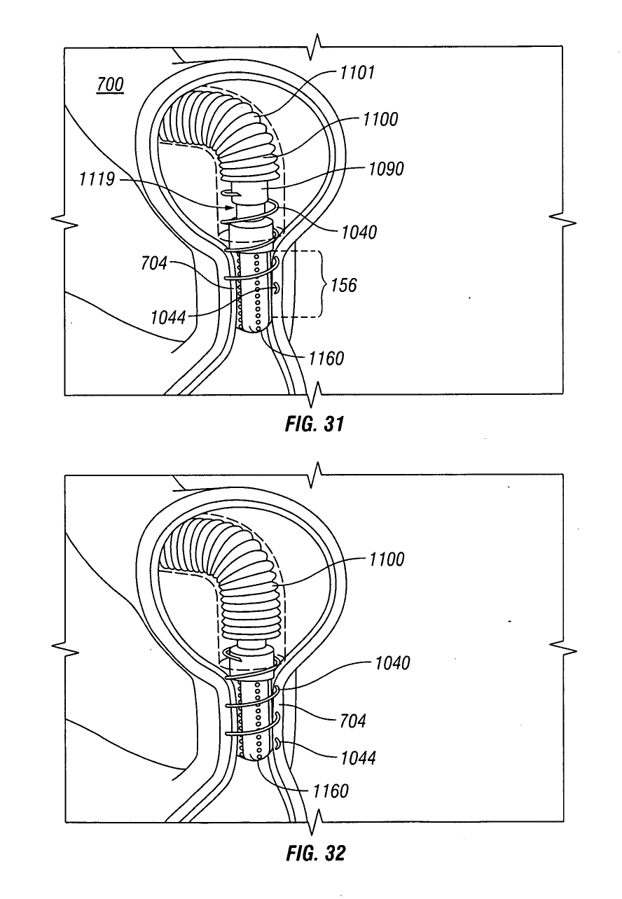

The suture driver 1100 may be bendable with the inner tube 1090, and may be further twistable to rotate the suture 1040. The corrugations 1101 (shown in FIG. 25, FIG. 31) of the suture driver 1100 may provide further flexibility and support for the suture driver 1100 when it is rotated or twisted by a user at the proximal end of the suture driver 1100. The suture driver 1100 may be made from a plastic, such as poly(tetrafluoroethylene) (PTFE) or other flexible chemically inert material and may be reinforced with stainless steel or other metal. It should be understood by persons of ordinary skill that other plastics and metals materials may be utilized for the suture driver 1100.

The body 1080 may further comprise an outer tube 1110 for housing the inner tube 1090, the suture driver 1100, and components of the proximal end 1002, such as the tissue gathering device 1020 and the suture assembly 1070. The outer tube 1110 may comprise an elongated cylindrical hollow tube that may receive the suture driver 1100 and the inner tube 1090 in an outer tube channel 1112 to, at least in part, seal the suture driver 1100 from body tissue and fluids. The outer tube 1110 may be made from a precision polyurethane tubing such as Tygothane® or other flexible, chemically-inert material.

An outer tube channel diameter 1114 may accommodate housing and actuation of the suture assembly 1070. The outer tube channel diameter 1114 may accommodate rotation of the suture driver 1100 and include a clearance between inner walls of the outer tube channel diameter 1114 and outer surface of the suture driver 1100.

The outer tube 1110 may be configured to couple at a proximal end to the handle assembly (shown exploded in FIGS. 7 and 8) and to couple at distal end to a suture connector 1060 (as shown in FIG. 18). The outer tube 1110 may be bendable with the suture driver 1100 and the inner tube 1090, and may be configured to allow the suture driver 1100 to twist or rotate within the outer tube channel 1112.

The inner tube 1090, the suture driver 1100, and the outer tube 1110 may each comprise a length spanning from a proximal end to a distal end of each tube 1090, 1100, and 1110. The lengths of each tube may be coordinated with each other to accommodate operation of the second gastric reduction device 1000. Generally, outer tube 1110 may comprise the shortest of the three tubes 1090, 1100, and 1110 to allow for the suture driver 1100 to extend out from the outer tube for positioning the tissue gathering assembly 1070.

The inner tube 1090 may comprise a length generally longer than the either the suture driver 1100 or the outer tube 1110. By example, to accommodate an average adult human, the length of the inner tube 1090 may comprise a value of 97 cm (38.2 in.). This length of inner tube 1090 may also accommodate connection of the inner tube 1090 to a vacuum source. The lengths, by example, of the suture driver 1100 and the outer tube 1110 may comprise 81 cm (31.9 in.) and 72 cm (28.3 in.), respectively. It should be understood by persons of ordinary skill that these lengths may be varied to accommodate accessibility of the second gastric reduction device 1000 to patients of different sizes.

### Handle Assembly 1006

At the proximal end 1004 of the second gastric reduction device 1000, the handle assembly 1006 may comprise a handle member 1120, a first insert 1130, a second insert 1140, and an actuator 1150. In some embodiments, the handle assembly may provide a mechanism for the operator to manipulate the second gastric reduction device 1000 and to actuate the suture assembly 1070.

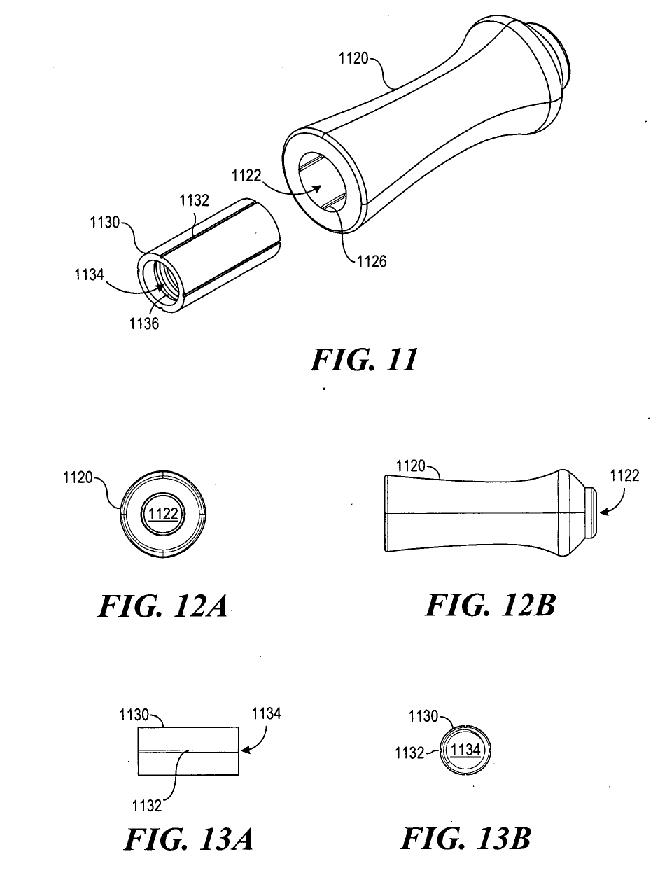

Turning now to FIGS. 10, 11, 12A and 12B, there are shown, in FIG. 10, a cross-sectional view of the handle assembly 1006, and in FIGS. 11, 12A and 12B perspective, rear and side views, respectively, of the handle member 1120. In some embodiments, the handle member 1120 may be generally hollow and configured to house, at least in part, the first insert 1130 and the second insert 1140.

The handle member 1120 may comprise a first handle bore 1122 extending along the device axis 1001 (shown in FIG. 8) through the handle member 1120 from an opening at a distal end for allowing passage of the suture driver 1100 and inner tube 1090. As shown in FIG. 10, the proximal end of the outer tube 1110 may couple at or near the distal end of the handle member 1120 at the first handle bore 1122. In some embodiments, the inner surfaces of the first handle bore 1122 may be glued to the outer surfaces of the outer tube 1110 so that a rim portion 1116 (also shown in FIG. 8) of the proximal end of the outer tube 1110 may abut the distal end of first insert 1130.

The handle member 1120 may have a curved shape or contours for accommodating a human grip so that the operator may manipulate the second gastric reduction device 1000. The handle member 1120 may be made from a plastic, such as acrylonitrile butadiene styrene (ABS) plastic resin, or other hard material.

Turning now to FIGS. 10, 11, 13A, 13B there are shown, in FIG. 10, a cross-sectional view of the handle assembly 1006, and, in FIGS. 11, 13A, and 13B perspective, front and side views, respectively, of the first insert 1130. The first insert 1130 may comprise a generally hollow cylindrical member for coupling the outer tube 1110 to the handle member 11120.

The first insert 1130 may fit into the first handle bore 1122 of the handle member 1120 from the proximal end of the handle member 1120 and may abut ledges 1124, shown in FIG. 10, in the inner surface of the first handle bore 1122, which may stop further translation of the first insert 1130 into the handle member 1120. In some embodiments, the outer surfaces of first insert 1130 may be glued to corresponding inner surfaces of bore 1122 of handle member 1120. Grooves 1132 may extend along an outside surface of the first insert 1130. The grooves 1132 may fit into tabs or ribs 1126 in the inner surface of the handle member 1120 for preventing relative rotation of the first insert 1130.

In some embodiments, the first insert 1130 may comprise a first insert bore 1134 extending through the hollow portion of the cylindrical member for providing a passageway for the suture driver 1100 and the inner tube 1090. The surface defining the first insert bore 1134 may comprise threads 1136 for receiving the second insert 1140. The first insert 1130 may be made from Delrin® or other similar material, including other resins, known by persons of ordinary skill in the art.

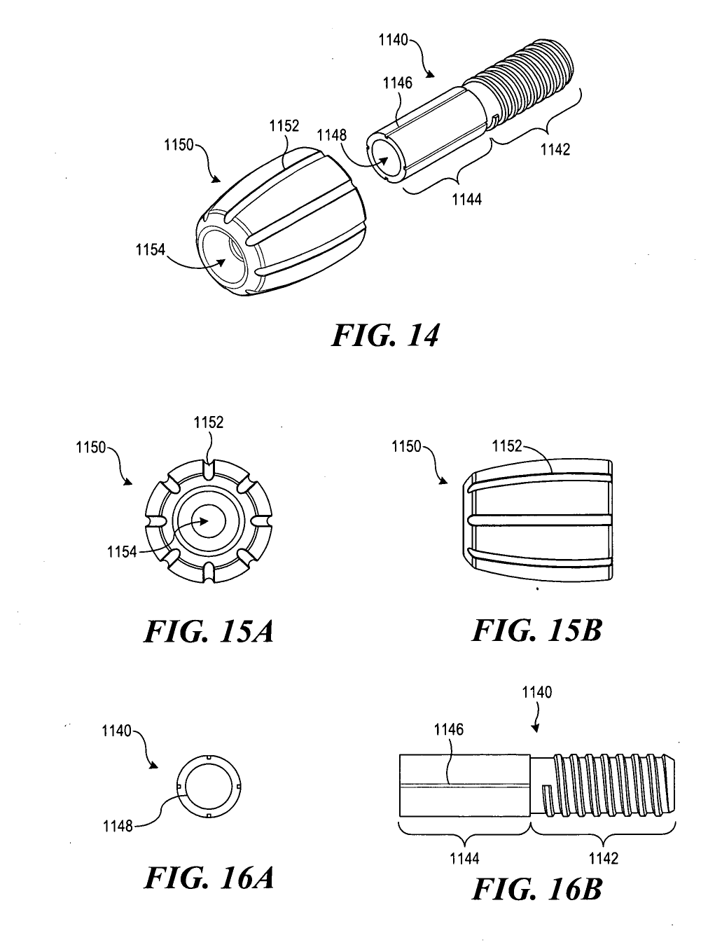

Referring now to FIGS. 14, 15A and 15B, there are shown perspective, rear, and side views, respectively, of the actuator 1150. In some embodiments the actuator 1150 may comprise a knob shaped member for actuating the suture driver 1100. Generally, the actuator 1150 may comprise a hollow cylindrical shape and have indentations 1152 extending along an outside surface of the actuator 1150 for providing a gripping surface for the operator to rotate the actuator 1150. The actuator 1150 may be manufactured from a plastic, such as acrylonitrile butadiene styrene (ABS) plastic resin, or other hard material.

An actuator bore 1154 may extend along the device axis 1001 through the center of the actuator 1150. The actuator bore 1154 may be configured and sized to fit receive, at least in part, the second insert 1140. The actuator 1150 may be configured to couple to the second insert 1140 for transferring a rotational force from the operator to a suture.

Turning now to FIGS. 14, 16A, and 16B, there are shown perspective, rear, and side views, respectively, of the second insert 1140. In some embodiments, the second insert 1140 may comprise generally a cylindrical hollow member for actuating the suture driver 1100. On at least a portion of an outside surface, the second insert may comprise a threaded first portion 1142. The threaded first portion 1142 of the second insert 1140 may threadably couple to the threads 1136 of the first insert 1130. The second insert 1140 may be made from a metal, such as aluminum.

The second insert 1140 may further comprise a second portion 1144 of the outside surface of the second insert 1140. The second portion 1144 may comprise a substantially smooth surface having one or more grooves 1146 that extend longitudinally along the device axis 1001 (as shown in FIG. 8). The second portion 1144 may be configured and sized to fit into the actuator bore 1154 of the actuator 1150.

Referring to FIG. 10, in some embodiments, the second portion 1144 may be glued to inner surfaces defining the actuator bore 1154. The inner surfaces of the actuator bore may also comprise one or more edge surfaces 1156 configured to prevent axial translation of second insert 1140 within the actuator bore 1154. The grooves 1146 of the second insert 1140 may receive tabs or ribs (not shown) that extend from the inner surface of the actuator bore 1154, as the second insert 1140 is fitted into the actuator bore 1154. The grooves 1146 may assist in preventing relative rotation of the second insert 1140.

In some embodiments, the second insert 1140 may further comprise a second insert bore 1148 for providing a coupling surface for the suture driver 1100. The second insert bore 1148 may extend longitudinally through the second insert 1140 substantially along the device axis 1001.

Referring to FIG. 10, the suture driver 1100 and the inner tube 1090 may pass at least partially through the second insert bore 1148. The suture driver 1100 may mount to the inner surface of the second insert bore 1148 to transmit driving force for the helical suture 1040 (as shown in FIG. 8) from the actuator 1150 to the suture driver 1100. The inner tube 1090 may pass through the second insert bore 1148 so that the inner tube 1090 may be coupled to a vacuum source (not shown).

### Suture Assembly 1070

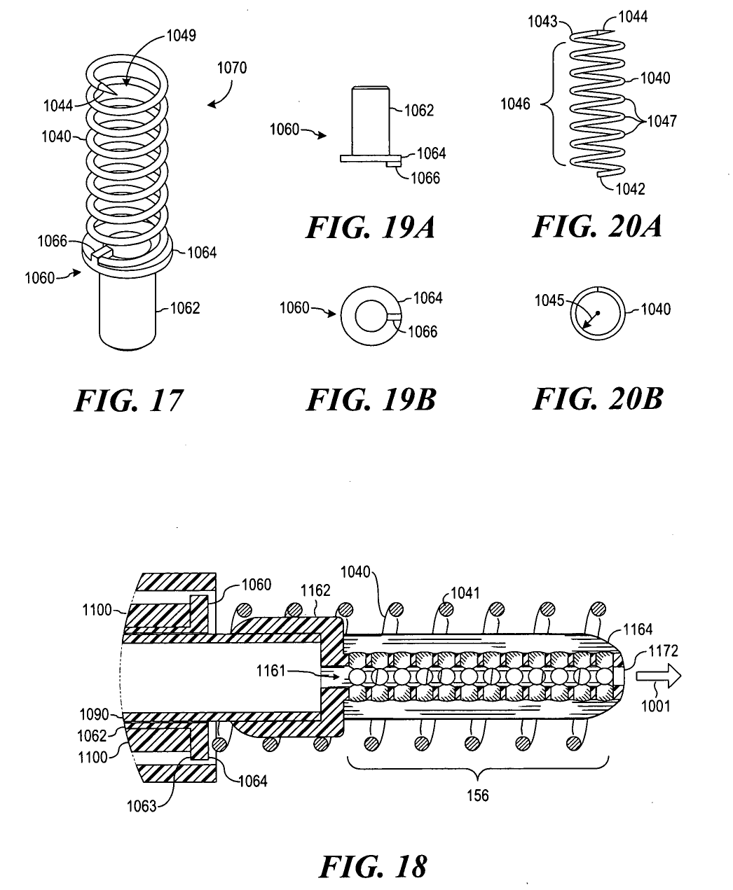

Referring now to FIG. 17, there is shown a perspective view of the suture assembly 1070, which may comprise suture connector 1060 coupled to helical suture 1040. The helical suture 1040 may be releasably coupled, in some embodiments, to the suture driver 1100 by the suture connector 1060. The connection between the helical suture 1040 and the suture connector 1060 may be configured so that the suture connector 1060 provides rotational force to the helical suture 1040 to advance the helical suture 1040 into the tissue of the stomach. Also, the suture connector 1060 may be configured to detach from the helical suture 1040.

Referring now to FIG. 18, there is shown a cross-sectional view of the suture assembly 1070 (as shown in FIG. 17) coupled to the tissue gathering assembly 1020. The helical suture 1040 may be coupled, in some embodiments, to the suture driver 1100 by the suture connector 1060. The tissue gathering assembly 1020 may provide a vacuum force to gather tissue near or to the second gastric reduction device 1000 for binding the tissue of the inner walls of the stomach. The suture assembly 1070 may provide a structure to form the tissue of the stomach into substantially a channel. In some embodiments, the suture assembly 1070 is at least in part actuated over the tissue gathering assembly 1020.

Referring now to FIGS. 19A and 19B, there are shown side and top views of the suture connector 1060. The suture connector 1060 may comprise generally a plug made of a first portion 1062 comprising generally a cylindrical shape, and a second portion 1064 comprising generally a plate shape having a radius generally larger than that of the first portion 1062. The suture connector 1060 may be made from stainless steel or other metal that is suitable for use within the body.

Referring to FIG. 18, the first portion 1062 of the suture connector 1060 may be configured to fit into the channel in the suture driver 1100 and over the inner tube 1090. In some embodiments, an outside surface of the first portion 1062 may couple to an inside surface of the suture driver 1100 at the distal end of the suture driver 1100 and, in certain embodiments, the coupling may be secured by glue. An edge portion of the distal end may abut an underside 1063 of the second portion 1064 of the suture connector 1060.

The second portion 1064 may cover at least partially the channel and edge portion of the suture driver 1100. In some embodiments, the second portion 1064 may provide a seal of the distal end of the suture driver 1100 to prevent tissue or fluids from entering the driver channel 1102 (as shown in FIG. 9) of the suture driver 1100.

Referring to FIG. 17, a top side of the second portion 1064 may comprise a substantially flat surface with a tab portion 1066, which may serve as a surface to couple the helical suture 1040 to the suture connector 1060. The tab portion 1066 may also provide a push surface for advancing the helical suture 1040. The helical suture 1040 may couple to the suture connector 1060 at or near the tab portion 1066.

### Helical Suture 1040

Turning now to FIGS. 20A and 20B, there are shown a side view, and a bottom view of the one embodiment for the helical suture 1040. The helical suture 1040 may be advanced into stomach tissue that has been brought into the suture area 156, shown in FIGS. 2 and 18. The helical suture 1040 may form a rigid structure for a gastric sleeve, creating a lumen or channel for ingested material to pass.

The helical suture 1040 may comprise a proximal end 1042, a distal end 1043, and a body portion 1046. The helical suture 1040 may be manufactured using standard techniques already well-known in the art. The helical suture 1040 may be constructed from stainless steel, though other suitable substantially rigid materials that are apparent to persons of ordinary skill in the art may be used.

The body portion 1046 may be comprised of multiple turns 1047 formed from the body portion 1046. Each turn may comprise a coil of a substantially helical shape comprising the helical suture 1046. The multiple turns 1047 may be spaced substantially evenly apart and sloped substantially in parallel forming a helix having a substantially constant pitch and a substantially constant radius of curvature. The multiple turns 1047 may extend from the distal end 1043 to the proximal end 1042.

In some embodiments, the helical suture 1040 may be formed from a material which allows for spring-like compression and expansion, whereby after expansion or compression of the helical suture 1040 it may return to its original condition upon the release of load. In other embodiments, the helical suture may be formed from a material that allows for compression or expansion of the radius of curvature, the pitch of one or more turns, or the overall length of the helical suture. Once fastened to the tissue of the stomach, the active gastric forces tending to distort the shape of the helical suture 1040 would be radial or axial in relation to the helical suture 1040 to cause generally expansion of the structure of the helical suture 1040.

The helical shape formed by the body portion 1046 may be that of a right-handed helix or a left-handed helix. The helical suture 1040 shown in FIG. 17 is generally that of a right-handed helix. Should a left-handed helical needle be required then the configuration the second gastric reduction device 1000 may be accommodated to correspond to the orientation of the multiple loops of a left handed helical needle. For example, at least the direction of threading in the first insert 1130 and second insert 1140 may be reversed to accommodate driving a left-handed helical suture.

The helical suture 1040 may further comprise a generally circular cross-sectional area 1041, as shown in FIG. 18, which may be uniform across the length of the suture body portion 1046, but may vary at the proximal end 1042 and the distal end 1043 of the helical suture 1040. The distal end 1043 of the helical suture 1040 may comprise a suture tip 1044. The suture tip 1044 may comprise a tapered shape wherein the cross-section 1041 of the suture tip 1044 increases from a point to a shape to match the cross section 1041 of the body portion 1046. The suture tip 1044 may further comprise a steep point or a shallow point depending on the needs of surgery.

The proximal end 1042 may comprise a cross-section similar to the cross-sectional area 1041 or in some embodiments may be varied to increase a contact area between the proximal end 1042 and the top surface of the suture connector 1060. Increasing the contact area may provide more surface area for coupling the helical suture to the connector 1060.

The length of the suture body portion 1046 may be taken as the distance along the suture body portion from the point of the tip 1044 to the proximal end 1042. The radius of curvature “r” may be substantially uniform across the multiple loops so that the needle body portion forms a helical of substantial uniform width. Other dimensions such as the helical pitch and the helical width may be configured to fit over the inner tube 1090. Further, these factors may vary according to the needs of a medical procedure and of a patient.

The helical suture 1040 may comprise a suture channel 1049 having a radius 1045. The suture channel 1049 may be formed from the loops 1047 of the helical suture 1040. In some embodiments, the radius 1045 may correspondence with the radius of curvature of the helical shape of the helical suture 1040. The radius 1045 may provide for creating a lumen or channel from tissue gathered to the second gastric reduction device 1000. Once fastened to the gathered tissue, the helical sutures 1040 may form a semi-permanent gastric sleeve.

The radius 1045 of the helical suture 1040 may be larger than the diameter of the desired lumen of tissue to accommodate for a desired a depth of penetration of the stomach tissue. The depth of penetration may be chosen so that the helical suture penetrates at least partially into the gastric muscle walls. Tissue binding may be more secure and durable at the muscular level. If the depth of penetration is too shallow, the bound mucosal (non-muscular) tissue may not hold and the gastric sleeve may breakdown.

In some embodiments, the diameter of the lumen may comprise a range of 8-12 mm (0.315-0.743 inches). In experiments performed, a lumen diameter of 10 mm (0.394 in.) was successfully used. The ranges given here are in no way intended to be limiting of what diameter of lumen may be incorporated or accommodated by the devices 100 and 1000 disclosed herein. The depth of penetration and its relation to the radius 1045 will be discussed further below in reference to the vacuum shaft 11162.

There may be certain advantages to using a helical suture in the embodiments disclosed herein. A helical suture allows tissue to be bound internally, allowing for a desired depth of penetration that will create a firm and lasting hold of the gastric sleeve. A helical suture may be advanced in a single continuous motion, which assists the operator in completing the procedure expediently. A helical suture may allow the suture to be stable and durable, because, at least in some embodiments, the depth of penetration is uniform and constant along the length of the helical suture. A helical suture is stronger than other thread materials and may be less likely to cut or slice tissue along the length of the helical suture.

In some embodiments, it may be desired that the helical suture 1040 be composed of nitinol (nickel titanium) or other similar material. Nitinol may comprise a material that may retain its original shape after being compressed or distorted. In some embodiments, the helical suture 1040 will be twisted to have a smaller diameter when the helical suture 1040 is housed within the outer tube 1110, but allowed to expand to its desired shape when the helical suture 1040 is deployed.

### Vacuum Shaft 1160

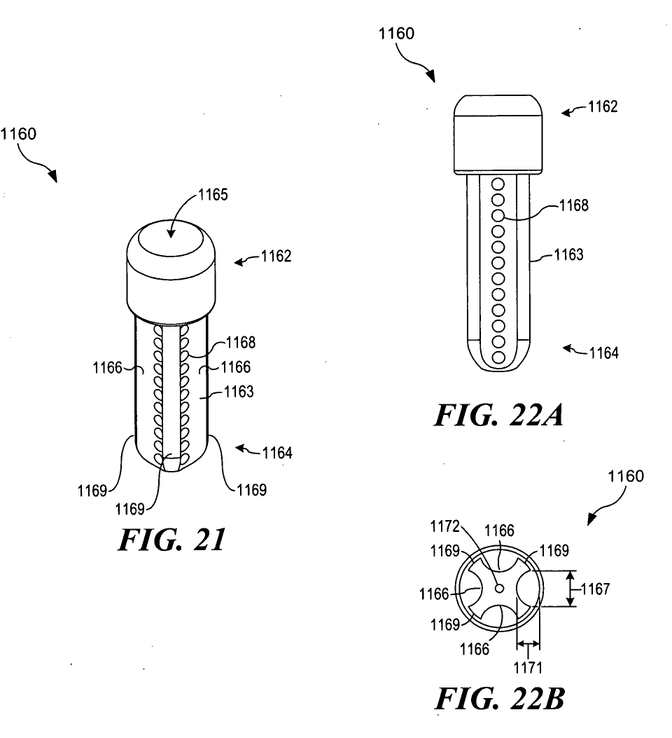

Turning now to FIGS. 21, 22A and 22B, there are shown a perspective, a side, and a bottom view of one embodiment for the vacuum shaft 1160. The vacuum shaft 1160 may comprise a vacuum connection end 1162 and a shaft distal end 1164. The vacuum connection end 1162 may be configured to receive and couple to the inner tube 1090 for transmitting a vacuum force to the walls of the stomach.

The shaft distal end 1164 may comprise a generally hollow cylindrical shaft portion 1163 having a vacuum channel 1161 extending longitudinally along the device axis 1001 generally through the center of the shaft portion 1163. The length of the shaft portion 1163 from the vacuum connection end 1162 to the shaft distal end 1164 may be configured to accommodate the formation of a lumen or channel in the stomach.

The vacuum connection end 1162 and the shaft distal end may be constructed as one integrated piece. The vacuum shaft 1160 may be manufactured from polycarbonate or other suitable material known by persons of ordinary skill in the art.

Extending longitudinally along the device axis, of the vacuum shaft 1160 may comprise one or more sections 1166. Each section 1166 may comprise a wall of the shaft portion 1163 extending along the device axis 1001. The one or more sections 1166 may surround the entire circumference of the shaft portion 1163 to face generally transverse to the longitudinal axis of the vacuum shaft. In some embodiments, the one or more sections may be configured to apply an equal circumferential force to the surrounding tissue of the stomach to draw the stomach to the second gastric reduction device 1000 to gather the tissue to the suture area 156.

In some embodiments, each section 1166 may comprise a curved surface 1167; for example the surface of the sections 1166 shown in FIG. 22B comprise a concave surface that curves inwardly toward the vacuum channel 1161. The curvature of the surface of each section 1166 may accommodate receiving the tissue of the stomach and the formation of the stomach into folds, as described below in FIGS. 25-34.

The curved surface 1167 may comprise a section depth 1171. The section depth 1171 may be defined as distance between an inner most portion of curved surface 1167 and substantially an outer edge of the vacuum connection end 1165 when viewed from the bottom view of the vacuum shaft 1160 shown in FIG. 22B. The section depth 1171 may comprise the depth at which the helical suture 1040 may penetrate the tissue that has been suctioned to the second gastric reduction device 1000.

Referring again to FIG. 20A, the radius 1045 of the helical suture 1040 may be configured to penetrate the tissue gathered to the second gastric reduction device 1000 at or near the section depth 1171. In some embodiments, the section depth 1171 may comprise a range of 3.0-5.0 mm (0.12-0.2 in.). Experiments have successfully achieved a desired amount of tissue at depths of 4.1 mm (0.16 in.). The ranges and examples of section depth given here are in no way intended as limiting the scope of penetration depths that the embodiments disclosed herein may be able to accommodate.

Referring now to FIGS. 18, 21, 22A, and 22B, there are shown, in FIG. 21, a perspective view of the vacuum shaft 1160, and FIGS. 22A and 22B, a side view and a bottom view, respectively of the vacuum shaft 1160. The curved surface 1167 of each section 1166 may be flanked on at least one side by one or more extensions 1169. Each section 1166 may also include one or more vacuum apertures 1168 for allowing the suction force to be transmitted to the inner walls of the stomach. Each aperture 1168 may, in some embodiments, comprise an opening, a slot or an inlet that may be configured to allow air or other fluids into the vacuum shaft 1160 in order to draw tissue by suction to each section 1166. Each vacuum aperture 1168 may be configured to allow simultaneous and equal distribution of vacuum forces, but may also allow for selectively applied vacuum to one area of gastric tissue at a time.

In some embodiments, the extensions 1169 may serve as partitions or arms to guide tissue as it is being suctioned to each section 1166. For example in FIG. 21, the extensions 1169 may flank an arrangement of vacuum apertures 1168 so that when tissue is drawn to the vacuum shaft 1160, the extensions 1169 guide at least a portion of the tissue to the vacuum aperture 11168.

Each extension 1169 may extend away from each aperture 1168 to create an open bowl or parabola. In some embodiments, the extensions 1169 may operate in pairs by flanking each side of each section 1166 so that tissue forms into folds at each section 1166. The folds of tissue may generally follow the contours of the curved surface 1167 and the extensions 1169.

In some embodiments, the extensions 1169 may reach past the radius of the vacuum connection end 1162 to increase the surface area that may guide the tissue around the vacuum shaft 1160. It is to be understood that the extensions may be retracted to accommodate deploying the helical suture 1040 over the vacuum shaft 11160.

Each vacuum aperture 1168 may extend from an outer surface of the section 1166 into the vacuum channel 1161. Each vacuum aperture 1168 may be positioned within the curved surface 1167 for drawing tissue to the outer surface of the vacuum shaft 1160. In some embodiments each aperture 1168 may be positioned at the deepest point of the curved surface 1167.

In some embodiments, each section may comprise an array of apertures 1168. Each aperture 1168 may comprise a shape, such as a generally circular shape. The apertures may be arranged in a line extending along the shaft portion 1163 of the vacuum shaft 1160.

It will be understood that the shape and arrangement of apertures may be varied to adjust the operation of the vacuum shaft 1160 in drawing tissue to the vacuum shaft 1160 by suction forces. The apertures may comprise slots, such as those shown and described in FIG. 1. The slots may extend in a linear, curved or spiral fashion. The slots may further be arranged in parallel or at offset angles from each other.

The vacuum channel 1161 may be connected to the inner tube 1090 by a coupling between the vacuum connection end 1162 and the inner tube 1090. The coupling may comprise gluing the distal end of the inner tube 1090 to the vacuum connection end 1162, but other methods known by persons of ordinary skill may be used as well. The vacuum connection end 1162 may comprise a generally hollow member having a vacuum connection bore 1165 a radius larger than the radius of the shaft portion 1163 and smaller than the radius of at least the turns of the helical suture 1040.

The inner tube 1090 may be configured to fit into the vacuum connection end 1162 and abut at least some inner surfaces of the vacuum connection bore 1165. The coupling between the inner tube 1090 and the vacuum connection end 1162 may form a seal for transmitting vacuum force and allowing suctioned air and other fluids from passing from the apertures 1168 into the vacuum port of the inner tube 1090.

A guide wire (as shown in FIG. 1 as guide wire 118) may be used in conjunction with the second gastric reduction device 1000, in a similar manner and fashion, as described in relation to the first gastric reduction device 100. The vacuum shaft 1160 may be configured with a guide wire aperture 1172, as shown in FIGS. 18 and 22B. The guide wire aperture 1172 may be configured to open the vacuum channel 1161 of the vacuum shaft 1160 for passage of the guide wire through the inner tube 1090 to the proximal end of the second gastric reduction device 1000. The guide wire aperture 1172 may be configured with a diaphragm-type seal to prevent loss of vacuum pressure within the vacuum shaft 11160.

It should be understood by persons of ordinary skill in the art that components of the second gastric reduction device 1000 may be interchanged with the components of the first gastric reduction device 100. For example, the suction tube 126, described above, may be substituted or modified to operate with the second gastric reduction device 1000.

### Operation Positions of Second Gastric Reduction Device 1000

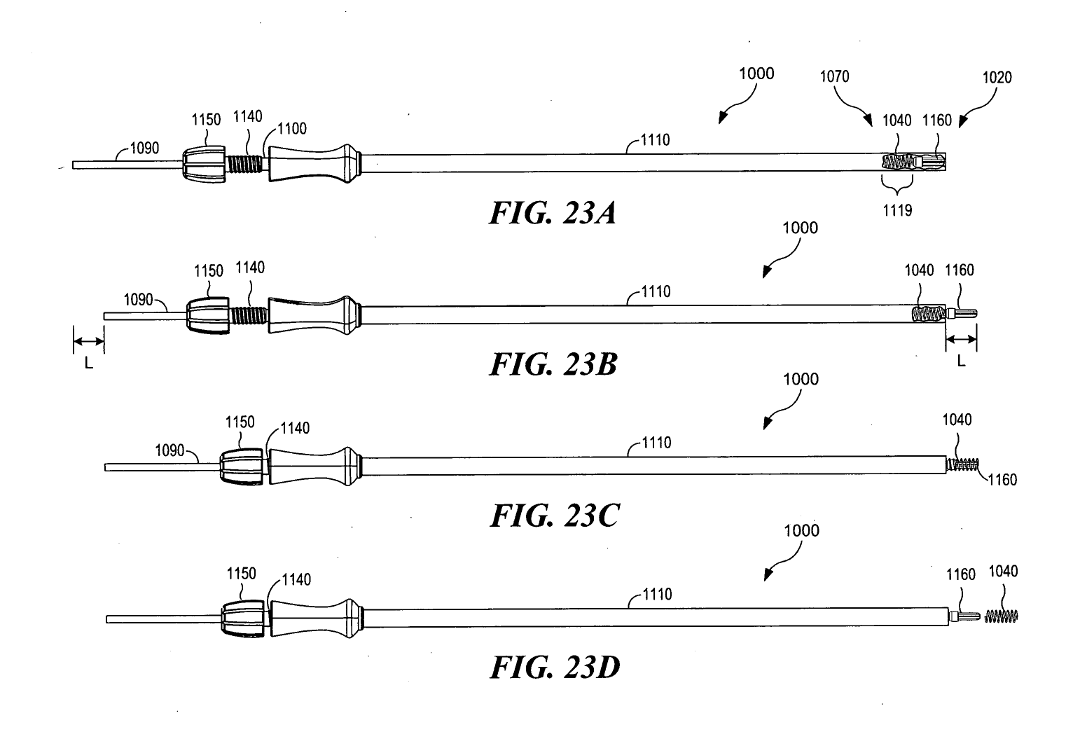

Turning now to FIGS. 23A, 23B, 23C, and 23D, there is shown a sequence of four operational positions for the second gastric reduction device 1000. The method of using the second gastric reduction device 1000 in relation to a human body will be described in greater detail below for FIGS. 25-34, below. In FIG. 23A, the second gastric reduction device 1000 is shown in a first position for initial insertion of the second gastric reduction device 1000 into an orifice of a living organism, such as the mouth of a human patient.

In this first position, the tissue gathering assembly 1020 and the suture assembly 1070 may be retracted within the outer tube 1110. During insertion through an orifice or tract of the patient, the second gastric reduction device 1000 may remain in the first position to prevent tissue from dragging or tearing on the working elements of the second gastric reduction device 1000.

In the first position, the second insert 1140 may remain disengaged from the first insert 1130 (not shown). In this position, the actuator 1150 and the second insert 1140 may be free to slide or translate over the inner tube 1090.

Also, in the first position, the helical suture 1040 may be positioned generally near the vacuum shaft 1160 in a suture housing area 1119 by moving the actuator 1150 along the inner tube 1090 so that the helical suture 1040 is translated toward the vacuum shaft.

In FIG. 23B, the vacuum shaft 1160 of the tissue gathering assembly 1020 may be progressed out of the outer tube 1110 to place the second gastric reduction device 1000 in a second position for applying a force to body tissue to gather tissue to the second gastric reduction device 1000. The vacuum shaft 1160 may be positioned to receive air or fluids through the one or more apertures 1168 (as shown in FIGS. 21 and 22A).

To transition from the first position, the operator may slide the inner tube 1090 a distance “L” relative to the outer tube 1110 to advance the vacuum shaft 1160 from the outer tube 1110. The distance L that the inner tube is advanced may comprise an adequate clearance for the vacuum shaft 1160 from the outer tube 1110 to prevent structural interference from the outer tube 1110 during tissue gathering and tissue suturing. Generally, the distance L may equal substantially the length of the vacuum shaft 1160.

In FIG. 23C, the helical suture 1040 is deployed out from the outer tube 1110 in a third position for engaging tissue from the stomach that has been suctioned to the device. The operator may move the actuator 1150 so that the second insert threadably engages the first insert 1130 (not shown). The operator may rotate the actuator 1150 so that the helical suture 1040 is advanced over the vacuum shaft 1160. In practice, the tissue of the stomach may have been positioned at the vacuum shaft 1160 so that the helical suture 1040 penetrates the tissue to attach the helical suture 1040 at the desired depth as it is rotated and translated over the vacuum shaft 11160.

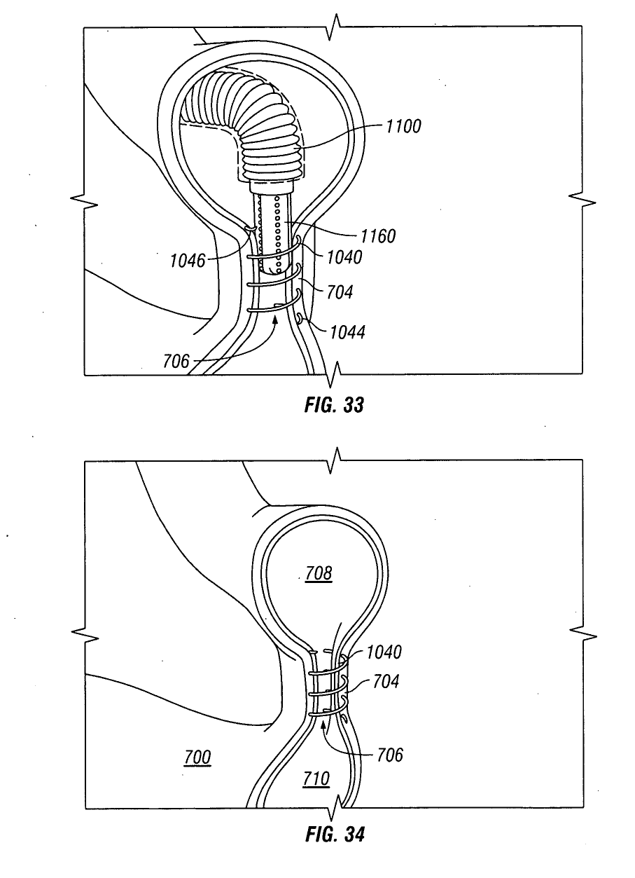

In FIG. 23D, the second gastric reduction device 1000 is pulled away from the attached helical suture 1040 in a fourth position. The operator applies a pulling force to break the coupling between the suture driver 1100 and the helical suture 1040 at the suture connector 1060, as described hereinafter with respect to FIG. 33. The vacuum shaft 1160 may be slid out from the suture channel 1048. The second gastric reduction device 1000 may be extracted from the body of the patient.

### Other Implements to Assist the Operator

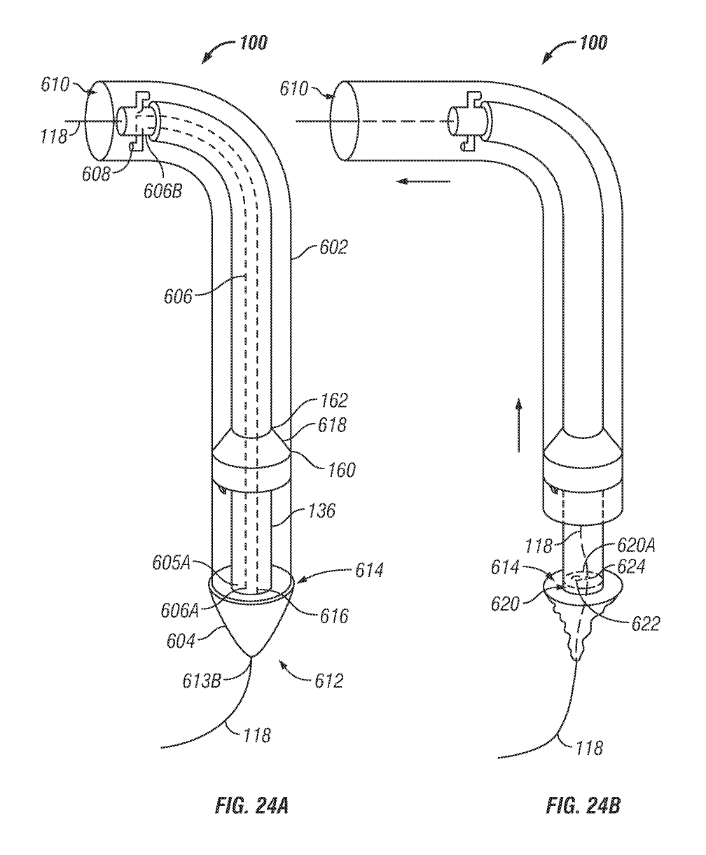

Referring to FIGS. 24A and 24B, the first gastric reduction device 100, shown in FIG. 1, may also include other implements which can assist the operator in reducing trauma associated with either inserting the first gastric reduction device 100 in the gastric system or extracting the first gastric reduction device 100. It is to be understood that such other implements may be interchangeably used with the second gastric reduction device 1000.

For example, an introducer sheath 602 may be utilized to provide a smoother surface upon insertion. The introducer sheath 602 comprises a flexible, hollow cylindrical tube with a passageway 610. The introducer sheath may be slid over the gastric device to cover it, at least partially. The introducer sheath may have a uniform diameter wide enough to fit in the passageway 610 the outer rotational shaft 104, the needle housing 128, and the suction tube 126. The introducer sheath 602 may be coated on its outside surface with a substance, such as silicon, to reduce friction between the first gastric reduction device 100 and the tissue. The introducer sheath 602 may be flexible enough so that as it passes with the other parts of the gastric device it bends and maintains the same relative position.

In some embodiments, the outer tube 1110, shown in FIG. 7, of the second gastric reduction device 1000 may be utilized in the same or similar way as the introducer sheath 602. The outer tube 1110 may protect the suture driver and house the suture assembly and tissue gathering assembly.

In conjunction with the introducer sheath 602, a tapered tip balloon 604 may be utilized to dilate the esophagus and provide for the insertion of the first gastric reduction device 100 to at least the diameter of the introducer sheath 602. The tapered tip balloon 604 may comprise a standard commercially available esophageal or pyloric balloon used to dilate structures and configured to have a tapered end 612 and a blunt end 614, giving the tapered tip balloon 604 a generally conical shape. The blunt end 614 may be configured to connect to the suction tube 126 and may further comprise an air inlet 616 to allow the tapered tip balloon to be blown up or deflated. In addition, the blunt end 614 may also comprise a guide wire entrance 613A (not shown) to allow the endoscopic device to pass from the blunt end to the tapered end and out of the balloon at a guide wire exit 613B.

The tapered tip balloon 604 may be coupled to the tube body 136 of the suction tube 126 in place of the tube tip 134. In some embodiments, the distal end of the suction tube 126 may be partially capped to seal the vacuum, except that sealed openings may allow passage of a balloon tube 604 and passage of the guide wire tube 119 (not shown). The tapered tip balloon 604 may also be coupled to the balloon tube 606 which may pass through the cavity 120 of the inner vacuum tube 110 from the distal end 112 to the proximal end 110.

The balloon tube 606 may comprise a distal end 606a which couples to the tapered tip balloon 604 and a proximal end 606b which couples to a balloon port 608 located on the proximal end 110 of the inner vacuum tube 102. The balloon tube 606 provides a separate and isolated channel for the insufflation and deflation of the tapered tip balloon 134. The balloon port 608 may couple to an air source (not shown) which may either inflate or deflate the tapered tip balloon 604 depending on what is needed.

In some embodiments, the tube body 136 of the suction tube 126 may be configured with a balloon retraction portion 620 (as shown in FIG. 6B) which may comprise an area located at the distal end of the suction tube 126 that is sealed from the vacuum source but is configured to allow the balloon tube 606 to pass and the guide wire tube 119 to pass. The balloon retraction portion 620 may comprise an inset portion of the suction tube 126 which cap and seals the suction tube 126 at cap 620A. The cap 620A is set into the suction tube 126 and sized to allow the tapered tip balloon 604 to be stored within it when the tapered tip balloon 604 is deflated. The cap 620A may also comprise balloon tube opening 622 and guide wire opening 624; these may be configured to allow passage of the balloon tube 606 and the guide wire 118 or the guide wire 118. Deflation of the tapered tip balloon 604 into the balloon retraction portion 620 may prevent the deflated tapered tip balloon 604 from obstructing gastric tissue as it is being suctioned.

### Method for Reducing Gastric Size

Referring now to FIGS. 25-34, certain aspects of this invention disclose, in one or more embodiments, methods to reduce gastric size. Generally, these methods may accomplish reduction of the size of the stomach to provide less space for foods and optimally reduce caloric intake capacity to the patient. In some embodiments, the methods disclosed here involve the use of a tissue gathering device, such as a suction device to gather a portion of walls of a stomach; the gathered portion of stomach walls are then bound together by stringing a suture through them and thus narrowing the passage of food through that portion of the stomach. By example, the suction device used in these methods may be the first gastric reduction device 100 or the second gastric reduction device 1000 disclosed in this description. Without limiting practice of the method to a particular device, these methods will be described in reference to the second gastric reduction device 1000 disclosed in this description. However, these methods may utilize other devices, which may accomplish the same or similar steps disclosed. Additionally, the methods disclosed here can optionally be used in conjunction with other gastric reduction procedures, depending on the medical needs of the patient.

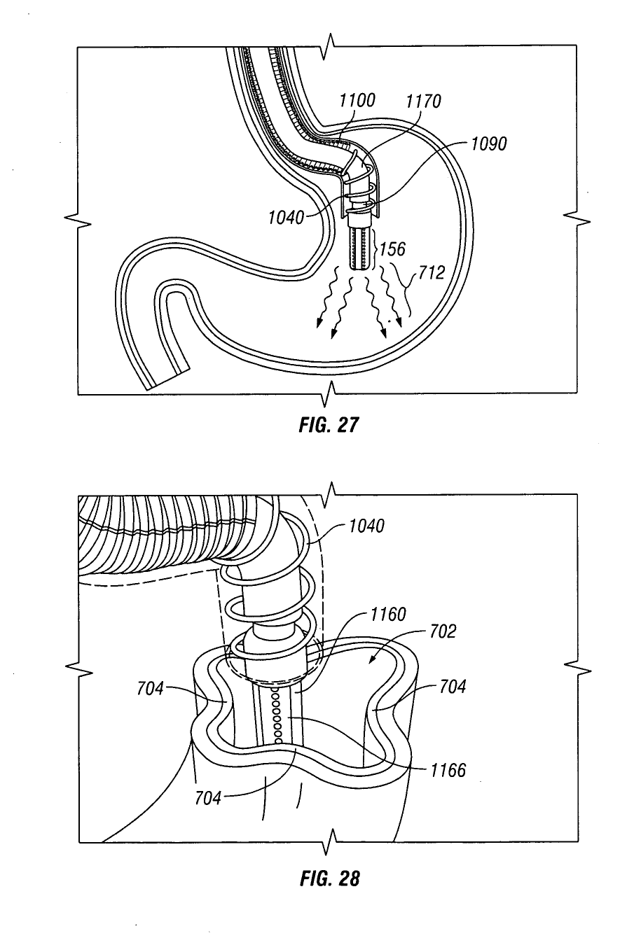

In certain FIGS. 25-34, certain parts have been cut away or hidden for clarity or for greater access to parts positioned out of view because of other parts. In FIGS. 27-33, the suture connector 1060 has been cut away to show the underlying structure. These illustrations are not intended to limit the device shown in any way.

To begin, the operator or other care supervisors may pre-select the configuration of the second gastric reduction device 1000 based on the anatomy and treatment needs of the patient. The operator or other care supervisor may determine necessary dimensions of the gastric reduction device to accommodate the given patient.

For instance, the operator may determine the length of the outer tube, taking into consideration the distance that the vacuum shaft 1160 and suture, such as the helical suture 1040 may extend when in the suture position, depending on the length of the esophageal tract and the size of the stomach of the patient.

The operator may determine the length of the suction tube 126 and the corresponding length of the suture, for instance the helical suture 1040 used in some embodiments in the second gastric reduction device 1000, or alternatively, the length of the helical needle 154 and the thread suture 520, used in some embodiments in the first gastric reduction device 100. The length of the suture may establish the quantity of the tissue to be bound and the amount in the reduction of gastric size desired. In addition, the operator may select the placement of the device relative to the portion of the stomach proximal to the esophagus. Also, the operator may optionally choose to utilize the introducer sheath 602 and taper tip balloon 604 to reduce trauma to the patient, as described in FIGS. 24A and 24B.

After standard pre-surgical preparations, including standard sedation techniques, the method shown in FIG. 25 may begin with placing a guide wire 118 (not shown) into the patient to assist in the placement of the second gastric reduction device 1000. An endoscopic device, not shown, may be slid over the guide wire 118 and into the patient's stomach 700. Such a technique is known in the art as endoscopy and generally refers to the direct or indirect assessment of an organ using a device with means to transmit visual images from the interior surfaces to the operator. The endoscope may be used to provide guidance to the operator in placing the second gastric reduction device 1000. In addition, the operator may utilize other indirect observational techniques such as fluoroscopy, taking advantage of radio-opaque elements of the second gastric reduction device 1000, to position the helical suture 1040 in a favorable position to complete the procedure.

Referring now to FIGS. 25-34, there are shown illustration of a method for reducing the volume of the stomach at least one embodiment. Once the operator has assessed where within the stomach the second gastric reduction device 1000 will be placed or the vicinity of where the operator has pre-selected for the placement of the second gastric reduction device 1000, the second gastric reduction device 1000 may be inserted transesophageally into the stomach of the patient. In certain embodiments, the second gastric reduction device 1000 may be advanced over a guide wire (not shown) by inserting passing the second gastric reduction device 1000 over the guide wire.

The second gastric reduction device 1000 may pass through the esophagus in the retracted position, shown in FIG. 25. In some embodiments, as shown in FIG. 23B, the second gastric reduction device 1000 may be inserted, in the second position, having the tissue gathering assembly 1020 extended from the distal end of the outer tube 1110. The second gastric reduction device 1000 may be configured so that the vacuum shaft 1160 is at least partially advanced from the outer tube 1110. In this position, the helical suture 1140 is coupled to the suture driver 1100 at suture driver connector 1060

As shown in FIG. 26, the operator may transesophageally position the second gastric reduction device 1000 in the stomach pouch 702 at a predetermined depth relative to the top of the stomach. The operator may position the second gastric reduction device 1000 such that the suture area 156 of the distal end 1002 is proximally near the portion 704 of the stomach walls that the operator has determined will be bound. Markings similar to markings 164 (shown in FIG. 1) may be positioned on the second gastric reduction device 1000 to assist the operator to place the vacuum shaft 1160 at the required depth.

The vacuum shaft 1160 may be advanced into the stomach 700 along a longitudinal axis of the body, extending from the head of the patient to the toe in a caudal direction. The vacuum shaft 1160 may be oriented so that the vacuum apertures generally face the wall portions 704 to provide efficient application of vacuum forces. Curvatures in the anatomy of the stomach 700 may necessitate some angular deflection of the vacuum shaft 1160 from the longitudinal axis of the body in order to orient the vacuum apertures.

As shown in FIG. 27, an endoscope 1170 may be passed over the inner tube 1090 and within the suture driver 1100 to position the endoscope 1170 or other observation device to illuminate and detect the inner walls of the stomach. The endoscope 1170 may provide a light source to illuminate the interior of the stomach (shown as wavy lines 712 in FIG. 27). The operator may also utilize fluoroscopic means and any radio-opaque elements, such as the helical suture 1040 and the suture drive connector 1060, to position the second gastric reduction device 1000.

In some embodiments, an introducer sheath 602 or a tapered tip balloon 604 may be utilized. As shown in FIGS. 24A and 24B, the introducer sheath 602 would be fitted onto the first gastric reduction device 100 prior to insertion into the patient and it would slide over the device 100 from the proximal end 402 of the outer rotational shaft 104 to the distal end 404 and over the helical needle housing 128 and suction tube 126. In conjunction with the introducer sheath 602, the tapered tip balloon may be utilized. The second gastric reduction device 1000 may be pre-configured to accept the tapered tip balloon 604 and may replace the tube tip 134. Prior to insertion and in conjunction with the installation of the introducer sheath 602, the tapered tip balloon 604 may be inflated through its balloon tube 606 connected to the balloon port 608.

Referring now to FIG. 28, once in position, the second gastric reduction device 1000 may apply continuous suction into the stomach cavity 702. By example, suction may be applied at a pressure of about 80 mm Hg (3.15 in. Hg) for approximately two minutes. This is amount of suction may collapse the portions 704 of the stomach walls toward the second gastric reduction device 1000, drawing or urging the portions 702 into a suture area 156 (shown in FIG. 27). In some embodiments, the sections 1166 of the vacuum shaft 1160 may draw one or more stomach wall portions 704 to each section 1166 of the vacuum shaft 1160. Each stomach wall portion 704 may form a fold in the stomach tissue, where each fold comprises a bend or doubling over of the stomach wall portion 704.

The folds of stomach wall portions 704 may form a channel or lumen 706 around the circumference of the vacuum shaft 1160. Suction force applied through the vacuum shaft 1160 may create one or more folds in the wall portions 704 to collapse the at least a part of the stomach.

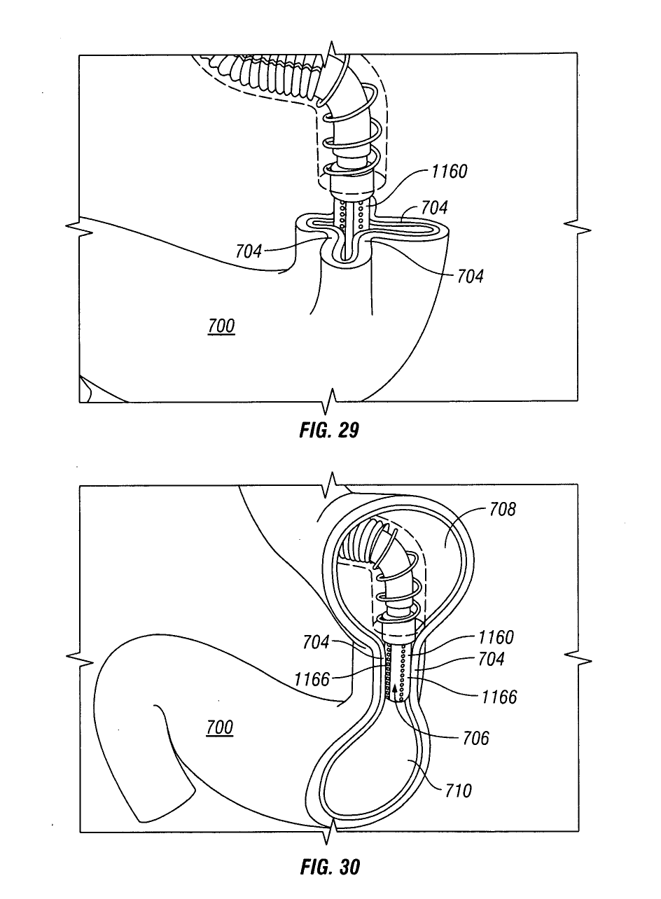

Referring now to FIGS. 29 and 30, the entire circumference of the stomach pouch 702 may be drawn by suction force to the vacuum shaft 1160. The wall portions 704 may be brought so that each stomach wall portion 704 makes contact with the vacuum shaft 1160.

In some embodiments, one or more guides (not shown) may assist in positioning the stomach wall portions 704 and forming the channel 706. In other embodiments, the extensions 1169 (shown in FIGS. 21 and 22B) may function as guides for forming the channel 706. The channel 706 may connect an upper pouch 708 of the stomach with a lower pouch 710, where generally the upper pouch 708 has a relatively smaller volume than the lower pouch 710.

Referring now to FIG. 31, the suture area 156 may be the area near the plurality of vacuum apertures 1168 in which the portions 704 of the stomach walls will be engaged and bound by the second gastric reduction device 1000. The suture area 156 defines the volume of space near the vacuum shaft 1160 where the second gastric reduction device 1000 may extend the helical suture 1040 into the stomach wall portion 704. Generally, the suture area 156 may comprise a generally cylindrical shape that generally follows the shape of the vacuum shaft 1160. The vacuum shaft 1160 allows a lumen or channel to be formed through the suture area 154 where stomach tissue may be generally prevented from entering.

To ensure that the proper amount of stomach tissue has been gathered into the suture area 156, the operator may use the guide wire (not shown) comprising an endoscopic device 1170 or other known direct or indirect observational methods.

Referring now to FIG. 31, once tissue has been gathered within the suture area 156, the operator may advance the helical suture 1040 from the suture housing area 1119 by turning, screwing, rotating or twisting the suture driver 1100 at its proximal end, which may extend outside the patient protruding from the mouth of the patient. The operator may utilize the handle assembly 1006 (as shown in FIG. 6A) and its coupling to the suture driver 1100. The direction of rotation of the suture driver 1100 may depend on whether a left-handed or right-handed helical suture 1040 is installed. The helical suture 1040 shown in FIGS. 25-34 is that of a right-handed helical structure. Thus, the operator may rotate the suture driver 1100 in a clockwise direction when looking towards the distal end 1002 of the device 1000 to advance the helical suture 1040 into the gastric tissue. The suture driver 1100 may rotate relative to the inner tube 1090.

As the helical suture 1040 is advanced by the operator, the tip 1044 may pierce the wall portions 704 of the stomach tissue and descend into the tissue in a direction towards a distal end of the vacuum shaft 1160. The helical suture 1040 may coil at least partially through the portion 704 of the stomach walls. As shown in FIG. 32, the advancement of the helical suture 1040 may continue until the helical suture 1040 is fully in place and fastened into the portions 704 of the stomach walls.

The helical suture 1040 may be advanced to pierce the one or more of the muscle layers of the stomach. In some embodiments, the helical suture 1040 may not pierce the outer layers of the stomach. The turns of the helical suture 1040 may be set into the portions 704 at a pre-determined penetration depth, as discussed in FIGS. 20A and 20B.

Referring to FIG. 32, when the helical suture 1040 has been advanced to a pre-determined length, the tension or drag may assist in anchoring the helical suture 1040 within the bound wall portions 704. In some embodiments, an anchor (not shown) may be deployed at one or more ends of the helical suture 1040 to keep the suture fixed to the wall portions 704 and prevent the helical suture 1040 from travelling or retracting out from the tissue.

In some embodiments, the tip 1044 of the suture may comprise a tear mitigation feature for preventing the sharp tip of the helical suture 1040 from tearing due the sharp tip rubbing against the wall portion tissue during normal movement of the muscles and walls of the stomach. In one embodiment, the tip 1044 may be detachable to allow the proximal end of the helical suture to be generally blunt. In another embodiment, the tip 1044 may be positioned so that at least a portion of the tip 1044 protrudes from the surface of the wall portions 704.

Referring to FIG. 33, once the helical suture 1040 is anchored into the wall portions 704, the operator may deploy the helical suture 1040 to disengage the helical suture 1040 from the suture driver 1100. At the proximal end, the attachment portion 1046 of the helical suture 1040 may be removed from the suture connector 1060 (shown in FIGS. 17 and 25) by retracting the helical suture 1040 to break the adhesive connection between the suture connector 1060 (cut away in FIGS. 27 through 31) and the helical suture 1040. This breakage may be facilitated by the drag force tending to keep the helical suture 1040 embedded within the wall portions 704

The operator may proceed to discontinue the suction applied to the vacuum shaft 1160, if it has not already been stopped. The operator may extract the vacuum shaft 1160 by using the handle assembly 1006 (not shown) to pull the vacuum shaft 1160 from the lumen 706. By binding portions 704 of the stomach walls forming a gastric sleeve of substantially uniform width and diameter, the operator may substantially reduce the total volume of the stomach.

Referring now to FIG. 34, once the vacuum shaft 1160 is extracted, the operator may remove the second gastric reduction device 1000 from the stomach, esophagus, and mouth of the patient by pulling the second gastric reduction device 1000 out. The helical suture 1040 may remain fastened and embedded in the wall portions 704, forming the channel 706. In some embodiments, the channel 706 connects the upper pouch 708 to the lower pouch 710 of the stomach 700.

The helical suture 1040 may remain fastened to the wall portions indefinitely, for as long as the weight loss effects of a reduced volume gastric space are desired, or until the integrity of the channel deteriorates. In some embodiments, the helical suture 1040 may be retracted or disengaged from the wall portions 704 by returning to the helical suture 1040 and rotating the helical suture 1040 in an opposite direction than that required to advance the helical suture 1040 originally.

Having thus described the present invention by reference to certain of its preferred embodiments, it is noted that the embodiments disclosed are illustrative rather than limiting in nature and that a wide range of variations, modifications, changes, and substitutions are contemplated in the foregoing disclosure and, in some instances, some features of the present invention may be employed without a corresponding use of the other features. Many such variations and modifications may be considered desirable by those skilled in the art based upon a review of the foregoing description of preferred embodiments. Accordingly, it is appropriate that the appended claims be construed broadly and in a manner consistent with the scope of the invention.

1. An apparatus for reducing stomach volume, the apparatus comprising:
a tissue gathering assembly, wherein the tissue gathering assembly is configured to apply a force to inner portions of a stomach to collapse the inner portions toward the tissue gathering assembly; and
a suture assembly moveably mounted to the tissue gathering assembly, wherein the suture assembly is configured to advance into the inner portions of the stomach to form a gastric sleeve between a first pouch and a second pouch in the stomach.

2. The apparatus of claim 1, wherein the tissue gathering assembly comprises a vacuum shaft having one or more apertures positioned around a circumference of the vacuum shaft, wherein the apertures allow air to flow to apply a suction force to the inner portions of the stomach.

3. The apparatus of claim 2, wherein the vacuum shaft comprises two or more sections that extend along a longitudinal axis of the shaft, and wherein at least one aperture is positioned in each section.

4. The apparatus of claim 3, wherein each section comprises a concave surface for defining a penetration depth for the suture assembly, and wherein the at least one aperture of each section is positioned in the concave surface to draw the inner portions of the stomach to the curved surface to within a suture area.

5. The apparatus of claim 2, wherein the suture assembly comprises a suture for binding the inner portions of the stomach to form the gastric sleeve.

6. The apparatus of claim 5, wherein the suture comprises a helical shape, and wherein the suture is rotatable to engage the inner portions of the stomach.

7. The apparatus of claim 6, further comprising a suture driver, wherein the suture driver comprises a rotatable shaft coupled to the suture to advance the suture into the inner portions of the stomach.

8. The apparatus of claim 7, further comprising a releasable coupling between the suture driver and the suture, wherein the suture is released from the suture driver by applying a pulling force to the releasable coupling.

9. The apparatus of claim 8, wherein the suture driver is configured to position the suture within a housing for transportation of the suture driver from outside a human body to inside a cavity in the human body.

10. The apparatus of claim 8, wherein the suture drive is configured to position the suture over the vacuum shaft for penetrating the inner portions in the suture area.

11. The apparatus of claim 9, wherein the housing comprises an outer tube mounted over the suture driver.

12. The apparatus of claim 3, further comprising an inner vacuum tube connected to the vacuum shaft for applying a suction force from a vacuum source located at an external part of the body to the inner portions of the stomach.

13. The apparatus of claim 3, wherein each aperture comprises a slot extending longitudinally along at least a portion of the vacuum shaft.

14. An apparatus for forming a sleeve from surrounding internal body tissue:
a shaft extending along a longitudinal axis, wherein the shaft is configured to couple to a vacuum source for applying a vacuum force to the internal body tissue;
one or more sections extending along an outside surface of the shaft generally along the longitudinal axis; and
one or more vacuum apertures positioned within each section, wherein each aperture connects to the vacuum source for applying the vacuum force to the surrounding internal body tissue.

15. The apparatus of claim 14, wherein each section is positioned around a circumference of the shaft for applying a uniform vacuum force to at least a portion of the surrounding internal body tissue.

16. The apparatus of claim 14, wherein the one or more sections are positioned about a circumference of the shaft to face substantially transverse to the longitudinal axis for forming a sleeve from the body tissue.

17. The apparatus of claim 15, wherein the outside surface of each section comprises a concave surface, and wherein at least one aperture is positioned within each section.

18. The apparatus of claim 17, further comprising one or more arms generally extending from a side of each section to flank each section for guiding the internal body tissue to the at least one aperture positioned within each section.

19. The apparatus of claim 18, further comprising a suture member for binding the body tissue within a suture area.

20. The apparatus of claim 19, wherein the suture member comprising a helically shaped suture having a channel for forming the sleeve.

21. A method for reducing gastric volume, the method comprising:
providing a tissue gathering device, wherein the tissue gathering device is configured to apply a force to inner portions of a stomach to collapse the inner portions toward the tissue gathering assembly;
positioning the tissue gathering device within the stomach, wherein the stomach is accessed through an esophagus;
gathering tissue of the inner portions of the stomach to within a suture area, wherein the suture area comprises an area immediately surrounding the tissue gathering device, and wherein tissue gathering device forms a channel from the gathered tissue;
providing a suture near the suture area;
advancing the suture into the suture area to penetrate at least a portion of the gathered tissue; and
binding the gathered tissue to form a sleeve, wherein the suture comprises a structure for maintaining the shape of the sleeve.

22. The method of claim 21, wherein gathering tissue of the inner portion of the stomach further comprises applying a suction force to lower the pressure within the stomach and to draw the tissue towards the suture area.

23. The method of claim 22, wherein the tissue gathering device comprises a shaft extending within the stomach generally in a caudal direction, and wherein the shaft is connected to a vacuum source.

24. The method of claim 22, wherein the suture comprises a substantially rigid helical shape for penetrating the gathered tissue.

25. The method of 23, wherein the suture comprises a substantially rigid helical shape for penetrating the gathered tissue.

26. The method of 25, further comprising rotating the suture to fasten the suture to the gathered tissue.
